(19) United States
(12) Patent Application Publication
(10) Pub. No.: US 2003/0175952 A1
(43) Pub. Date: Sep. 18, 2003

(54) Materials and methods relating to increasing viral titre

(76) Inventors: David Darling, London (GB); Farzin Farzaneh, London (GB); Christopher Paul Hughes, Stockport (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/258,039

(22) PCT Filed: Mar. 22, 2001
(86) PCT No.: PCT/GB01/01261

(30) Foreign Application Priority Data
Apr. 18, 2000 (GB) 0009619.8
Feb. 14, 2001 (GB) 0103731.6

(51) Int. Cl. C12Q 1/70; A61K 39/21; C12N 7/02; C12Q 1/68; C12N 5/06; C12N 5/16

(52) U.S. Cl. 435/345; 424/208.1; 435/5; 435/239; 435/6

(57) ABSTRACT

The invention provides methods for increasing viral titre in a sample. The methods utilize specific binding members such as lectins and antibodies to bind the virus particles such that they can be concentrated. The invention further provides methods for isolating viral particles from a sample, e.g. blood. There is also provided materials and methods for targeting viral particles to particular tissues using antibodies or paramagnetic particles.

### FILED OF THE INVENTION

The present invention relates to materials and methods concerned with increasing viral titre. Particularly, but not exclusively, the present invention relates to novel methods allowing purification and concentration of retrovirus from packaging cell supernatant. The invention also relates to the application of these methods in the field of targeting specific tissues, treatment of disease states and for screening and diagnostics.

### BACKGROUND OF THE INVENTION

Retrovirally mediated gene therapy requires either, retroviral packaging cells releasing large numbers of infectious retroviral particles and/or methods for enhancing the efficiency of retrovirus/target cell interactions (and preferably both). Efforts to optimize the effective titre of retroviral vectors have tended to focus upon three strategies.

1) New vector constructs have been designed that allow for more efficient expression and packaging of retroviral vector RNA^{1,2}. These have been coupled with a new generation of packaging cell lines producing greater numbers of retroviral particles^{3,5}, which may also have wider target cell trophisms^{3,5,6} and be more resistant to inactivation, either by centrifugation⁶ or human complement^{5,6}.

2) Culture conditions for optimum retroviral vector particle production and efficiency of retroviral/target cell interactions have been improved. Retroviral production can be increased by “ping-pong” of mutually infectible packaging cells⁷, superinfection⁸, culture of target⁹ or packaging cells at 32° C.¹⁰, and in some cases by the addition of the histone deacetylase inhibitor sodium butyrate¹¹. Once secreted into the supernatant the probability of retroviral particles infecting a target cell is increased by incubation with polycations such as polybrene and protamine sulphate^{12,13}, complexing retroviral particles with liposomes or liposomal lipids¹⁴, flow-through of culture supernatant¹⁵ and low speed centrifugation of retroviral particles with their target cells^{9,10}. Target cells can also be made more receptive to infection by culturing them in phosphate depleted medium¹⁰, or by the addition of fibronectin and its truncated derivatives¹⁶.

3) Many of the above methods can be combined with concentrated or purified retrovirus. Reducing the supernatant volume whilst preserving the retroviral infectibility has been reported following the passage of retroviral supernatant through molecular weight cut-off filters^{9,17-20}, lyophilisation⁹ and co-precipitation with calcium phosphate²¹. However such reductions can be accompanied by the concentration of other components that may be inhibitory to infection²² or toxic to the target cells²¹. Such developments have resulted from the limitations of centrifugation as a method for purification of intact infectious retrovirus. The various versions of the “classical method” involve ultracentrifugation for up to 16 hours at 18000 g²³, and appear to work very well. However the volume of supernatant that can be purified is relatively small, the efficiency of recovery of infectious retrovirus is low, and the method works best with low titre retrovirus²³. Other variants still appearing in the literature use reduced speed centrifugation²⁴⁻²⁶ (to as little as 3000 g) whilst maintaining the time period, or increased g force and reduced duration¹⁹. Centrifugation does however have many attractions, it is easy to perform, and can reduce supernatant volume in the absence of copurification of inhibitors of infection. Centrifugal concentration has so many potential advantages that packaging cells for pseudotyped retroviral vector production have been designed with resistance to inactivation by centrifugal stress as a major consideration^{4,6}. Such VSV-G (vesicular stomatitis virus G) pseudotype vectors can be subjected to 50,000 g for 90 minutes^{6,27} resulting in up to 2000-fold reductions in volume with more than 70% recovery of infectious virus⁶.

In the absence of an optimized retroviral concentration protocol for vectors other than VSV-G pseudotypes, one cannot simply increase the number of retroviral particles applied to the target cells by using more supernatant. This is due to the balance between a retroviral vector half-life of 5-7 hours at 37° C. and the probability of it coming into contact with a cell target during that period. A retroviral particle traveling by Brownian motion alone is unlikely to travel more than 600 μm in one half-life²⁸. Thus, in a static culture of 1 ml as much as 90% of the retrovirus may be unavailable for infection. Since target cell availability is finite, one cannot use a sufficiently large number of cells in order to efficiently interact with all the available retrovirus.

### SUMMARY OF THE INVENTION

The present inventors have appreciated that there is a need for methods which will provide an increase in viral titre, thereby increasing the amount of virus available for infection. Thus in the example for retroviruses given above, to fully utilize the 90% of the retrovirus unlikely to contact the cells, the present inventors have designed methods which utilise binding members capable of binding to the retrovirus to form a complex. This complex can then be harvested efficiently from the packaging cell supernatant. The inventors have surprisingly found that the harvesting can be achieved by short-term low speed centrifugation which results in at least 1000-2000 fold increase in titre after only 200-fold reduction in volume.

Therefore, at its most general, the present invention provides a method for increasing viral titre from a sample comprising viral particles, said method comprising contacting said sample with a binding member capable of binding to the viral particles to form a complex; concentrating the complex, e.g. by centrifuging the sample; and, if necessary determining the viral titre.

The increase in viral titre does not result in an increase in the amount of virus in the sample. In fact, the exact amount of virus in the sample is not necessarily known and, with regard to the present invention, the increase in viral titre must be taken to be a function of both the amount and the likelihood of the virus infecting a target cell. Thus, the increase in virus titre in said concentration may be determined by infectivity of target cells.

For convenience, the following text illustrates the invention and aspects thereof by referring to retroviruses. However, it will be apparent to the skilled person that the invention can be applied to all viruses including retroviral pseudotype packaging cell lines, e.g. MoMulv vectors with vsvg pseudotype envelopes. For example, the inventors are particularly interested in the retroviridae family which includes ssRNA, positive sense, non-segmented genome, enveloped and DNA step in replication viruses. Subfamilies of Retroviridae include oncovirinac (i.e. Molony Murine Leukaemia virus) Lentivirinac (i.e. HIV and other lentiviral vectors) and spumavirinae.

The invention may however, be applied to other virus families such as Herpesviridae (dsDNA and enveloped virus), examples include Herpes simplex virus, Epstein Barr virus, Cytomegalovirus, Varicella-Zoster virus.

Other families include Adenoviridae (ds and non-enveloped viruses), examples include adenovirus and adenoviral vectors such as those based on Ad.5; Papovaviridae (ds and non-enveloped viruses) e.g. Simian vacuolating virus 40 and polyoma virus; Picornaviridae (ssRNA +sense, non-enveloped, non segmented viruses) e.g. enterovirus, poliovirus; Rhabdoviridae (ssRNA, −ve sense, non-segmented, and enveloped viruses) e.g. vesticular stomatitis viruses; Poxviridae (dsDNA, non-enveloped viruses) e.g. poxvirus (variola) and vaccinia. Other families groups to which the invention may be applied include the following Arenaviridae, Birnaviridae, Bunyaviridae, Caliciviridae, Coronaviridae, Filoviridae, Flavivirdae, Hepadnaviridae, Iridoviridae, Orthomyxoviridae, Paramyxoviridae, Paroviridae, Reoviridae, Togaviridae. This list in not intended to be exhaustive. As mentioned above, the following text for convenience refers to retroviruses including lentiviruses.

Following the method defined above, it may be desirable to separate the retrovirus from the binding member. However, the present inventors have identified a number of binding members which may be used in the above method and which do not necessarily require separation from the retrovirus in order for the retrovirus to maintain efficient infectivity. These are discussed below as separate aspects of the present invention.

For example, in a first aspect of the invention, the inventors have designed a method that uses a cheap readily available, particulate and dense substrate. When retrovirus in packaging cell supernatant is mixed with excess substrate it forms retroviral/substrate complexes that are dense enough to settle under gravity in static culture within the half-life of the virus. Such complexes can also be subjected to short-term low speed centrifugation in order to create the retroviral concentration if necessary. Using this particulate and dense substrate the inventors have found that the virus surprisingly remains infectious, does not require special treatment to facilitate its release and the non-toxic substrate can remain in culture long enough to allow for optimal retrovirus/target cell interaction^{14,29}.

Therefore, the present invention provides a method for increasing the retroviral titre in a sample, comprising adding to said sample a dense and particulate substrate capable of forming a complex with said retrovirus; centrifuging said sample so as to concentrate said complex; and determining the concentration of said retrovirus as determined by infectivity of target cells.

Preferably, the dense and particulate substrate is Pansorbin, a heat killed, formaldehyde fixed staphylococcus aureus, although other substrates such as Sansorbin may be used. It may be the case that other bacteria possess the same ability as Pansorbin and Sansorbin. This may be on the basis of fibronectin binding proteins (fnb) proteins resident on the surface of the bacteria and fnb type proteins may be expressed in different bacteria. Thus, it is within the capabilities of the skilled person to determine other suitable dense and particulate substrates given the teaching presented herein.

As described in detail below in the “Detailed Description”, the present inventors have demonstrated that retroviral particles shed from the murine fibroblast derived PG13 packaging cells (Gibbon Apc Leukaemia Virus [GaLv] envelope protein pseudotyped) can be efficiently concentrated as infectious retrovirus. PG13 derived retroviral particles spontaneously complex with heat-killed, formaldehyde fixed staphylococcus aureus (Pansorbin). These complexes can be centrifugally concentrated and the associated retroviral particles retain their capability to infect target cells without the need for prior measures to facilitate their release.

Kayman S. C. et al. (J. Virology. March 1999, p1802-1808) describes, amongst other things, the use of Pansorbin to deplete (or negatively enrich) supernatant of wild-type virus, although they are not concerned with increasing retroviral titre. However, in contrast to the present invention, the authors use an antibody against an epitope previously inserted into the env gene of the virus (SC258) and mixed this with the virus and Pansorbin on the basis that the Fc of the antibody would bind to protein A on the Pansorbin. The authors state that positive enrichment requires recovery of infectious virus from the bound state, and that standard conditions used to disrupt the antibody-antigen complexes are lethal to the retrovirus. In contrast, the present inventors have surprisingly determined that the complex of the retrovirus and Pansorbin does not need to be disrupted in order for the retrovirus to maintain infectivity. Thus, Pansorbin or other like particulate and dense substrates, e.g. Sansorbin, may be used to aid the increase in retroviral titre and no loss in concentration or even infectivity is lost as the subsequent disruption of the complex formed with the retrovirus can be avoided.

The present inventors believe that this observed binding, coupled with the preservation of the ability to infect, could be explained by the recently cloned fibronectin binding proteins (fnbA³² and fnbB³³) resident on the surface of the Staphylococcus aureus Cowan I strain used in Pansorbin. These proteins may interact with the murine fibronectin secreted by the NIH 3T3 cells upon which PG13 packaging cells are based, Fibronectin in the supernatant may then become associated with PG13 derived retroviral particles. Thus, it would appear that retrovirus is not directly interacting with Pansorbin, but rather associated via a fibronectin intermediary. This fibronectin interaction is known to promote retroviral infectivity rather than inhibit and may explain why the retrovirus remains infective when complexed to Pansorbin.

Although this method is highly effective with PG13, PA317 and GP+envAM12 derived retrovirus the results were less encouraging with other retrovirus such as the NIH3T3 based GP+E-86 and the human HT1080 based FLYRD18 and A13 packaging cells.

Therefore, the present inventors have designed a further method as a second aspect of the present invention, based on the premise that retrovirus can be captured from the supernatant by using antibodies directed against fibronectin. Thus, in accordance with the second aspect of the present invention, the binding member is an antibody or antibody binding domain directed against a protein associated with the virus. The antibody may be directed against a protein actually resident on the surface of the virus, or it may be directed against a protein associated with the virus, i.e. a protein that has a natural binding affinity for the retrovirus.

As the antibody binding member is not a dense substrate, it would not be possible to concentrate the complex formed using gravity in the static culture or short-term low speed centrifugation. Therefore, the second aspect of the present invention also provides the use of a capture agent for capturing the complex such that it may be concentrated. Examples of such capture agents include paramagnetic particles (PMP) (Promega; other suppliers include Dynal, Miltenyi Biotec) which may be concentrated using a magnet. Non-magnetic beads could also be used provided they are small, e.g. in order of 1 μm, and sensitive to low speed centrifugation. In the example provided herein, the antibody in question is directed against murine fibronectin and, using streptavidin coated Paramagnetic Particles conjugated with a polyclonal Rabbit anti-mouse fibronectin antibody, the inventors magnetically purify PG13 derived particles on the basis of their association with murine fibronectin. The inventors have shown that the antibodies may be bound to protein A via their Fc domain. Protein A may be bound to the PMP by biotin in order to complete the complex. Alternatively, a biotinylated polyclonal antibody may be used.

Thus, this second aspect of the present invention requires the use of a first coupling partner associated with the capture agent, e.g. streptavidin; and a second coupling partner associated with the antibody binding domain, e.g. biotin. The skilled person will be able to devise other coupling partners which may be used in association with the invention.

However, the method in accordance with the second aspect of the present invention is only applicable to murine packaging cells secreting fibronectin and producing virus e.g. retrovirus with fibronectin binding activity. Therefore, in order to provide a more universal strategy the inventors have adapted this procedure to use a lectin based affinity capture methodology which extends the useful range of this affinity capture strategies to other viral packaging cells e.g. retroviral packaging cells such as human HT1080 derived packaging cells. This is based on the observation that retroviral envelope proteins are glycosylated on a packaging cell specific basis, and that surface modifications to retrovirus shed from these cells reflects this fact.

Thus, in accordance with a third aspect of the present invention, the binding member is a lectin capable of binding to glycosylated proteins on the surface of the virus e.g. retrovirus. The inventors have appreciated for the first time that post-translational glycosylation modifications to the surface proteins, e.g. envelope protein, of the retroviruses may be utilised in this method without the need for release from the binding member in order to maintain infectivity. In the examples described below concerning this aspect of the present invention, the lectins used are Isolectin B₄ (BS-IB₄) isolated from Bandeiraea Simplicifolia (binding the α-Galactosyl groups absent in humans) or Succinyl-Concanavalin A which primarily binds the α-mannose modifications. Other lectins known to the skilled person that bind glycosylation sites may be used, e.g. PHA.

As with the second aspect of the present invention, a capture agent may be required so as to capture the lectin/retrovirus complex so that is may be concentrated either by gravity in the static culture or by short-term low speed centrifugation. Again, the capture agent is preferably paramagnetic particles.

With regard to both the second and third aspects of the present invention , the capture agents require a mechanism by which they can capture the complex (e.g. coupling partners such as antibody/retrovirus or lectin/retrovirus). This mechanism may be produced by coupling partners such as biotin/biocytin-avidin/streptavidin, receptor-ligand, antibody-antigen, etc. Thus, the complex may be designed such that it further comprises one member of a coupling partner, e.g. biotin, and the capture agent may be designed so as to comprise the other member of the coupling partners, e.g. streptavidin. In this way, when the complex and the capture agent are bought into contact, the coupling partners join (biotin-binds streptavidin) thereby linking the complex and the capture agent.

Other examples exist. For example, the present inventors utilise protein A which binds with the Fc region of an antibody. Thus, an antibody may be used that binds to the retrovirus by being specific for a protein resident on the surface of the retrovirus. Protein A may be biotinylated such that it is held on the capture agent (e.g. PMP coated with streptavidin), and brought into contact with the antibody/retrovirus. The Fc region of the antibody and protein A act as coupling partners and serve to bring the antibody/retrovirus complex and the capture agent (PMP) into contact. The skilled person will appreciate that protein G and protein L may equally be used instead of protein A.

With the use of coupling partners, e.g. a ligand, it is preferable to attach these to the capture agent, e.g. PMP, prior to their introduction into the vial supernatant. In this way, the ratio of capture agent/coupling partner to retrovirus complex in the supernatant can be optimised.

Finally the inventors have investigated a fourth strategy that may be applicable to all viral, preferably retroviral packaging cell types presently available (and those yet to be developed). In addition to responding to those packaging cell specific postranslational modifications of retrovirus, the inventors have investigated a more proactive approach involving introducing modifications to the retroviral surface. This approach utilizes the protein specific covalent coupling activity of succinimide esters, for example, biotin succinimide ester. Biotin modification of the surface proteins of packaging cells results in an infectious retrovirus that is efficiently captured by, for example, streptavidin PMPs.

Thus, the inventors have surprisingly found that coupling partners may be used to incorporate binding members onto the surface of the retrovirus. This is preferably achieved by using a succinimide ester derivative that is capable of co-valently coupling biotin to proteins on the surface of packaging cells. Retrovirus derived from these biotinylated cells affinity couples to, for example, streptavidin on a capturing agent.

Thus, in a fifth aspect of the present invention there is provided a method of modifying a viral particle so as to ease its capture from a sample comprising said modified viral particles so as to increase their titre, said method comprising the steps of incorporating a coupling partner on to the surface of a viral packaging cell so that viral particles derived from the packaging cell display said coupling partner on their surface.

The methods according to the present intention not only allow virus to be concentrated from a packaging cell supernatant to an increased titre, but also provide other advantages arising from the use of the binding partners described herein.

For example, the inventors have found that the retrovirus does not need to be separated from the binding surface in order to be infective. Further, the inventors have found that the capture agent/binding member/virus complex can be frozen. Although the thawing process will lose up to 50% activity of the retrovirus, this is considerably better than other known methods. In addition, because of the starting concentration of retrovirus following the methods of the present invention is high but in small volume, very small amounts of freezing solution, e.g. DMSO, are used as opposed to unconcentrated virus. This is extremely valuable in treating patients owing to the toxicity of DMSO.

The inventors have further appreciated that the complexes formed in accordance with the present invention may be used in the treatment of diseases. For example, the PMP/retrovirus complex may be used for in vivo targeting. Use of appropriate PMP would allow them to be isolated to a specific region by use of NMR to focus the required magnetic field.

Thus, the present intention also provides the use of complexes formed in accordance with the methods described above, in the preparation of medicaments for targeting virus, e.g. retrovirus to tissues in vivo. Further, the invention provides a method of targeting a retrovirus complex to a tissue within a human or animal (mammal) body, said complex comprising a retrovirus and a magnetic particle, said method comprising administering said complex to the human or animal body, and drawing the complex to the tissue using a magnetic field.

A further method of targeting tissues may also be provided which utilizes any spare binding capacity on the binding member or capture agent. For example, if the surface of the retrovirus has been modified in accordance with the fourth aspect of the present invention to carry biotin, it may be captured by streptavidin PMPs. However, the inventors have found that not all of the streptavidin will be bound. Thus, a biotinylated antibody directed to a particular tisse antigen may also be attached to the PMP via the streptavidin-biotin coupling partners. In this way, the antibody will bind to the specific tissue antigen thereby bringing the bound retrovirus into contact with the tissue. Alternatively, protein A may be used to bind the Fc domain of the antibody. The tissue antigen may be any known antigen specific for the tissue type selected for targeting. In one embodiment the tissue antigen may be a tumour antigen which would allow the retrovirus to be brought into contact with a particular tumour. The retrovirus may be further modified to carry foreign nucleic acid for use in gene therapy. This method has advantages in that the retrovirus does not need to be modified according to the tissue type targeted.

The inventors have also appreciated that the method according to the present invention may be used in the labeling or identification of virus. For example, capture agents and/or coupling partners described above may be used to identify or isolate viruses from biological samples such as blood, serum, urine, semen etc. HIV virus may be efficiently pulled out of blood samples. This may provide an HIV test of increased sensitivity (about 100 fold increase). Further, such a method may be used in association with other techniques used to treat biological samples. For example, capture agents and/or coupling partners may be used to remove virus from serum during dialysis.

Thus, in a further aspect, the invention provides a method of isolating or removing a virus from a sample (e.g. blood), said method comprising the steps of

(a) contacting said sample with a binding member capable of specifically binding to the virus so that the virus and binding member form a complex;

(b) contacting the complex with a capture agent capable of capturing said complex; and

(c) isolating or removing said complex and capture agent from the sample.

As described above, the capture agent may capture the complex via coupling partners such as biotin and streptavidin. If the capture agent is a PMP, it may be removed from the same along with its captured complex using a magnetic field. The capture agent may also be a solid support coated with a coupling partner such as streptavidin, over which the sample is passed. If the binding member is an antibody or lectin which is associated with a coupling partner such as biotin, it will couple to the solid support thereby removing or isolating the complex from the sample, e.g. blood, urine or serum.

Described herein are detailed procedures for each of the aspects of the present invention applied to both murine and human derived packaging cell supernatants.

As a further aspect of the present invention, there is provided a kit or use of a kit for carrying out a method of increasing retroviral titre from packaging cell supernatant in accordance with the methods described above; said kit comprising a binding member capable of binding to virus and optionally a capture agent and/or a coupling partner as defined above. The kit would also usefully comprise instructions for carrying out the method of the invention. Examples of components for a kit according to the present invention are paramagnetic beads, magnet, protein A-biotin complex, anti-murine fibronectin antibody anti-human fibronectin antibody, concanavalin A-biotin complex, BSI-B₄-biotin complex, biotin succinimide ester, and wash buffer.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE DRAWINGS

FIG. 1 Preincubation of retroviral supernatant with increasing doses of Pansorbin increases effective titre. The cfu/ml, of PG13 derived retroviral (IL-2/B7) supernatant was determines after incubation with formalin fixed Staphylococcus aureus. Aliquots of 5 mls of retroviral supernatant were either immediately titred (T=0, Control) or incubated for 2 hours at 4° C. in the absence of Pansorbin (T=2, Control) or with the indicated volumes of Pansorbin, after which serial dilutions of the mix were used to infect K562 cells. Each value represents the mean and standard deviation of triplicate colony counts.

FIG. 2 The duration of Pansorbin/retrovirus preincubation provokes a proportionate increase in effective titre. Aliquots of 5 mls of PG13 derived retroviral (IL-2/B7) supernatant were incubated in the absence of Pansorbin, or with 25 μl Pansorbin and serial dilutions of the mix were used to infect K562 cells at the times indicated. Each value represents the mean and standard deviation of triplicate colony counts.

FIG. 3 Centrifugal concentration of PG13 derived retroviral vector (IL-2/B7) supernatant/Pansorbin complexes. Retroviral supernatant was harvested, a sample taken, and serially diluted for immediate infection of K562 cells (T=0). The bulk of the supernatant was then divided into 50 ml aliquots and incubated for 3 hours at 4° C. with either no further addition (T=3), 250 μl Pansorbin alone (P) or 250 μl of Sansorbin (S). Serial dilutions of samples from T=3, P and S were then used to infect K562 cells. In addition 5 ml aliquotes of T=3 and P were subjected to further 0.45 μM filtration and used for infection (2nd Filter and P Filter). All samples were then centrifugally concentrated and the aspirated supernatant from T=3 and P used for titration (Cont Dep and Pdep). The residual supernatant in T=3 was mixed thoroughly (as if to resuspend a pellet) and the used for titration (Contconc). The remaining Pansorbin and Sansorbin sample pellets were resuspended in 10 ml of fresh medium, centrifuged once again, resuspended in a minimal volume of medium (180 fold reduction on starting volume) and taken for infection (Pconc and Sconc). Each value represents the mean and standard deviation of triplicate colony counts.

FIG. 4 Pansorbin mediated retroviral titre enhancement is not only effective on K562 cells. Retroviral supernatant derived from PG13 IL-2/B7 was harvested, divided into 50 ml aliquotes and incubated at 4° C. with either no further addition (T=3), 250 μl of Pansorbin (P). After 3 hours samples from all conditions were used to infect NB4, U937 and HeLa cells (T=3) The remaining volume of each Pansorbin/retrovirus sample was centrifugally concentrated as described and once again used to infect NB4, U937 and HeLa cells (Pconc). Each value represents the mean and standard deviation of triplicate colony counts.

FIG. 5 Pansorbin complexes with retrovirus independently of vector insert. Retroviral supernatant derived from PG13 RaRT was harvested and immediately used to infect K562, NB4, U937 and HeLa cells (T=0). The remaining 50 ml supernatant was incubated for 3 hours at 4° C. in the presence of Pansobin (P). After incubation, the samples were subjected to centrifugal concentration and used for infection (Pconc). Each value represents the mean and standard deviation of triplicate colony counts.

FIG. 6. The efficiency of Pansorbin mediated concentration varies with packaging cell type. Retroviral supernatant derived from GP+envAM12IL-2/B7, PA317IL-2/B7, FLYRD18pBabe.puro, FLYA13pBabe.puro and GP+E-86pBabe.puro was harvested and immediately titred (Control) on either human K562 or murine 32Dp210 myeloid cells. The remaining samples were divided into either 50 ml (FLYRD18, FLYA13 and GP+E-86) or 10 ml (GP+envAM12 and PA317) aliquotes and incubated at 4° C. with either 250 μl or 50 μl Pansorbin. After 3 hours samples were centrifugally concentrated as described and once again used to infect target cells (Pansorbin concentrate). Each value represents the mean and standard deviation of triplicate colony counts.

FIG. 7. Paramagentic particle mediated concentration of fibronectin associated PG13 derived retrovirus. The cfu/ml of PG13 derived retroviral (pBabe.puro) supernatant was determined before and after incubation with Paramagnetic Particles (PMPs). Aliquots of 5 mls of retroviral supernatant were either immediately titred (Control), or incubated for 2 hours at 4° C. with 2.5×10⁹ Streptavidin magnespheres (pre-conjugated with either Protein A-biotin alone or Protein A-biotin and Polyclonal Ig Rabbit anti mouse fibronectin). After magnetic concentration and washing as described, serial dilutions of the mix were used to infect K562 cells. Each value represents the mean and standard deviation of triplicate colony counts.

FIG. 8. Lectin/PMP mediated PG13 retroviral particle capture and concentration. The cfu/ml of PG13 derived retroviral (pBabe.puro) supernatant was determined before and after incubation with Paramagnetic Particles (PMPs). Aliquots of 5 mls of retroviral supernatant were either immediately titred (Control), or incubated for 2 hours at 4° C. with 2.5×10⁹ Streptavidin magnespheres (pre-conjugated with either the biotinylated lectin Isolectin B₄ (BSI-B₄), the biotinylated lectin Concanavalin A (ConA). After magnetic concentration and washing as described, serial dilutions of the mix were used to infect K562 cells. Each value represents the mean and standard deviation of triplicate colony counts.

FIG. 9. Lectin/PMP mediated FLYRD18 and A13 retroviral particle capture and concentration. The cfu/ml of FLYRD18 and FLYA13 derived retroviral (pBabe.puro) supernatant was determined before and after incubation with Paramagnetic Particles (PMPs). Aliquots of 5 mls of retroviral supernatant were either immediately titred (Control), or incubated for 2 hours at 4° C. with 2.5×10⁹ Streptavidin magnespheres (pre-conjugated with either the biotinylated lectin Isolectin B₄ (BSI-B₄), the biotinylated lectin Concanavalin A (ConA). After magnetic concentration and washing as described, serial dilutions of the mix were used to infect K562 cells. Each value represents the mean and standard deviation of triplicate colony counts.

FIG. 10a. Biotin succinimide ester/PMP mediated PG13 retroviral particle capture and concentration. The cfu/ml of PG13 derived retroviral (pBabe.puro) supernatant was determined before and after incubation with Paramagnetic Particles (PMPs). Aliquots of 5 mls of retroviral supernatant from untreated, DMSO incubated, or Biotin succinimide ester labeled packaging cells were either immediately titred (Control, DMSO, BiotinSE), or the supernatants from DMSO incubated or Biotin labeled packaging cells were incubated for 2 hours at 4° C. with 2.5×10⁹ Streptavidin magnespheres. After magnetic concentration and washing as described, serial dilutions of the mix (DMSOconc, BiotinSEconc) were used to infect K562 cells. Each value represents the mean and standard deviation of triplicate colony counts

FIG. 10b. Flow cytometric analysis of Biotin labeled packaging cells. PG13 cells were biotinylated as described and incubated overnight at 37° C. After harvesting supernatant for retroviral processing the cells were removed from the substratum as described and 1×10⁶ cells were labeled with Avidin FITC. The two profiles represent those cells incubated with carrier alone (DMSO, solid line) or those biotinylated (BiotinSE, dashed line) 24 hours prior to Avidin-FITC labeling. The mean fluorescence index (MFI) of the carrier control is 6.5 and that of the biotinylated cells is 2000.

FIG. 11. Biotin succinimide ester/PMP mediated FLYRD18 and A13 retroviral particle capture and concentration. The cfu/ml of FLYRD18 and FLYA13 derived retroviral (pBabe.puro) supernatant was determined before and after incubation with Paramagnetic Particles (PMPs). Aliquots of 5 mls of retroviral supernatant from untreated, DMSO incubated, or Biotin succinimide ester labeled packaging cells were either immediately titred (Control, DMSO, BiotinSE), or the supernatants from DMSO incubated or BiotinSE labeled packaging cells were incubated for 2 hours at 4° C. with 2.5×10⁹ Streptavidin magneSpheres. After magnetic concentration and washing as described, serial dilutions of the mix (DMSOconc, BiotinSEconc) were used to infect K562 cells. Each value represents the mean and standard deviation of triplicate colony counts.

FIG. 12 shows freezing and thawing preparations. Control unconcentrated retrovirus was frozen in 2-5 ml aliquots by placing at −20° C. Concentrated PMP captured retrovirus was frozen by the addition of three volumes of standard freezing mixture (10% DMSO, 20% FCS, final DMSO concentration: 7.5%) and placing at −20° C. Retroviral preparations were thawed as rapidly as possible, and an aliquot removed for testing and the remains returned to −20° C.

FIG. 13. Magnetic field mediated localization of retroviral infection of HeLa cells. Plate a) shows the toxic effect of puromycin selection of an uninfected culture, Plate b) demonstrates the even spread of infection in the absence of magnetic targeting whilst Plate c) shows efficient directed infection of target cells to specific regions of the culture as evidenced by the pattern of drug resistant cells surviving as a result of retroviral infection. Plate d) shows the original magnetic template used to direct the localized infection and demonstrates how closely the targeted infection mirrors the shape of the original template.

### DETAILED DESCRIPTION RELATING TO THE FIRST ASPECT OF THE PRESENT INVENTION

Incubation of PG13 Supernatant with Formalin Fixed Staphylococcus aureus (Pansorbin)

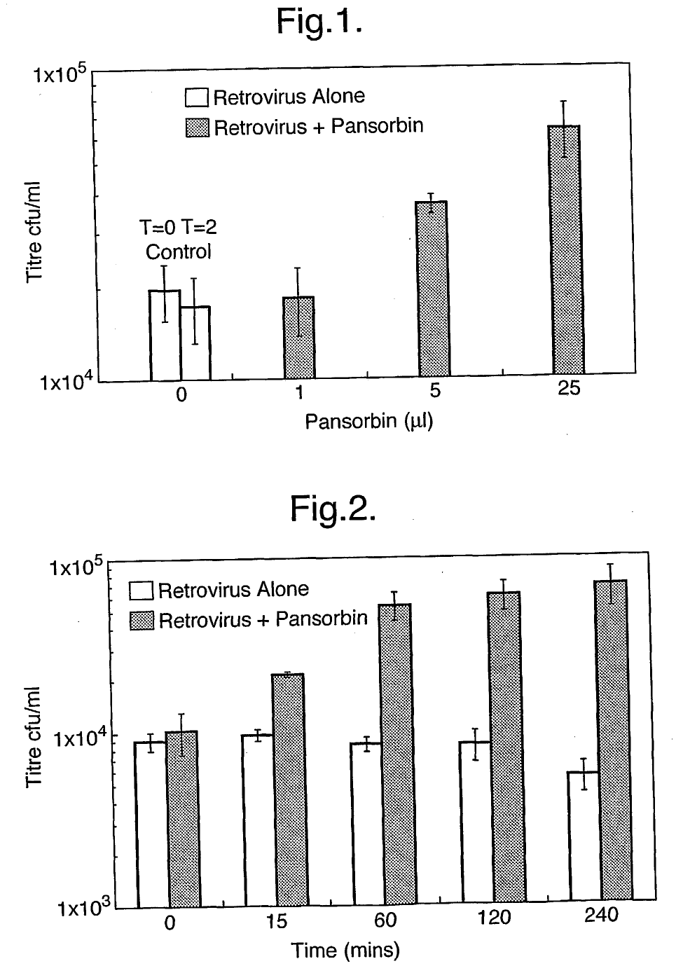

For gene therapeutic purposes we have been using the murine fibroblast derived PG13 packaging cells pseudotyped with the GaLv (Gibbon Ape Leukaemia virus) envelope protein³. A mixed population of these cells producing a MoMuLv based vector (pWZLIL2/B7M Fusagene³⁰, conferring resistance to Blasticidin S Hydrochloride and subsequently referred to as IL-2/B7) was titred on K562 cells using a soft agar colony counting assay. A representative example of the titre obtained (1.95×10⁴ cfu/ml) from this mixed population can be seen in FIG. 1 (T=0, Control). Though this titre is a substantial improvement on previous packaging cells this is still not efficient enough for our purposes.

The inventors thus decided to test a hypothesis that since both Galv pseudotype³¹ retrovirus and Staphylococcus aureus^{32,33} are know to adhere to fibronectin then retrovirus associated with a relatively large and dense bacteria may then be able to settle under gravity in culture and increase the localized concentration of retrovirus capable of infecting target K562 cells. A readily available source of cell culture compatible Staphylococcus aureus is heat-killed and formaldehyde fixed but still retains functional Protein A on its surface³⁴ and may thus retain other surface expressed protein activities.

FIG. 1 shows an experiment using PG13 derived IL2/B7 retroviral supernatant preincubated with Pansorbin. In the absence of Pansorbin the freshly harvested supernatant has a cfu/ml titre on K562 cells of 1.95×10⁴±4×10³/ml (T=0, Control). After 2 hours (T=2, Control) incubation at 4° C. in the absence of Pansorbin the cfu/ml is unchanged at 1.75×10⁴±4×10³/ml. However the presence of Pansorbin in the preincubation has a dose-dependent effect on the cfu/ml titre of the retrovirus. Thus up to 25 μl of Pansorbin preincubated for 2 hours with a PG13 derived retroviral vector incubation increases the effective cfu/ml titre on K562 cells by as much as 3-fold.

The effect of Pansorbin/Retrovirus Preincubation Duration on Infectivity

Using 25 μl of Pansorbin/5 ml retroviral supernatant the optimum length of preincubation was investigated (FIG. 2). A plateau of Pansorbin mediated increase in titre was reached after 2 hours incubation (5.3×10⁴±9.5×10³ cfu/ml) whilst the control cfu/ml titre in the absence of Pansorbin remained stable,(T=0, 9×10³±1.1×10³ cfu/ml: 2 hours 8.33×10³±1.7×10³ cfu/ml) but drops to 5.4×10³±1.1×10³ cfu/ml after 4 hours. In subsequent experiments an incubation time of 3 hours and 25 μl of Pansorbin/5 ml supernatant was routinely used. It is noticeable that the enhancement in cfu/ml titre after 2 hours in FIG. 1 using 25 μl of Pansorbin was 3-fold and under the same conditions in FIG. 2 it was more than 7-fold. This is consistent with other experiments (data not shown) and an average for this type of experiment is approximately a 5-fold cfu/ml enhancement.

Low Speed Centrifugal Concentration of Retrovirus After Complexing with Pansorbin

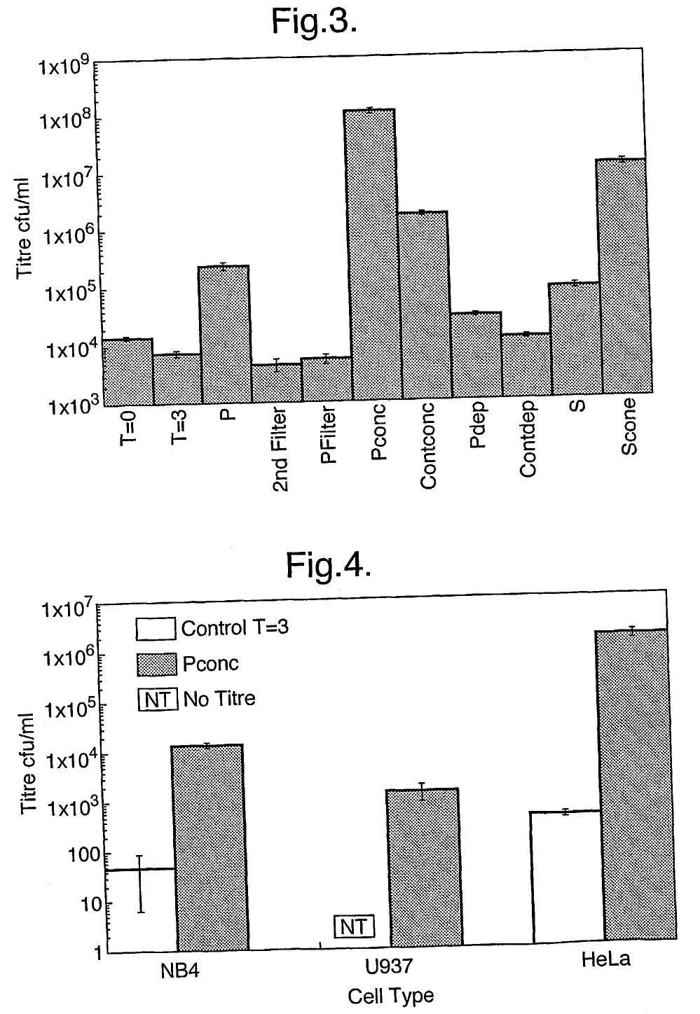

The experiments described in FIGS. 1 and 2 were performed on small volumes of supernatant, but in order to be a practical method scaling up is required. The Pansorbin specific settling of retrovirus under gravity alone suggested that low speed centrifugation may allow concentration and purification of retrovirus from packaging cell supernatant. The inventors thus incubated 50 ml samples of retroviral supernatant with 250 μl of Pansorbin and attempted to concentrate the retrovirus; FIG. 3 shows the results of a typical experiment.

The control titre before incubation is roughly equivalent to the analogous titre in FIGS. 1 and 2, (1.4×10⁴±7.5×10² cfu/ml), after 3 hours preincubation at 4° C. this has dropped by half to 7.2×10³±7.3×10² cfu/ml indicating that this may be the upper advisable limit of incubation time. Once again a 3 hour incubation with Pansorbin enhances the effective cfu/ml from 1.4×10⁴ to 2.3×10⁵ (ie 16-fold). The efficiency of this part of the process is a compromise between the optimum binding time, reduced retroviral infectivity, and nonspecific binding of retrovirus or Pansorbin to the container. This may explain why the preincubation alone is more efficient in the scaled up version after incubation for only 3 hours. In order to demonstrate that the virus is physically complexed with the Pansorbin, 5 ml aliquots were removed from the incubation mix, filtered once again through a 0.45 μm filter, and immediately titred. A second filter of the retrovirus after 3 hours incubation in the absence of Pansorbin reduces the titre by 38% (from 7.2×10³ cfu/ml[T=3] to 4.4×10³ cfu/ml [2nd Filter]). The same treatment performed on Pansorbin incubated samples (P) reduces titre by more than 97% (P:P Filter 2.3×10⁵ cfu/ml:5.4×10³ cfu/ml). Thus more than 97% of retroviral titre can be removed from the coincubation by filtration alone.

Low speed centrifugation of the remaining 45ml of Pansorbin retrovirus mix, washing once, and resuspending in 250 μl of fresh medium (180-fold volume reduction) prior to titration results in a cfu/ml of 1.1×10⁸ cfu/ml (Pconc), representing an increase in cfu/ml of 7.8×10³-fold. The effective titre detected in the concentrate is substantially higher than one might expect when compared with the control supernatant alone. This anomaly is less striking when the concentrate is compared with supernatant incubated with Pansorbin and titred without centrifugation (P conc, 1.1×10⁸ cfu/ml:P, 2.3×10⁵ cfu/ml), though it still achieved a 473 fold increase in titre for only an 180 fold reduction in volume. An infection titration performed with the top 25 ml of the supernatant from the centrifuged samples (Pdep) demonstrates that not all the retroviral infectivity has pelleted alongside the Pansorbin, there still remains a titre in the supernatant of 2.7×10⁴ cfu/ml. This may represent retrovirus that did not bind to the Pansorbin, or retrovirus associated with Pansorbin that did not pellet under low speed centrifugation.

As a control, centrifugation of retroviral supernatant was also performed after 3 hours incubation in the absence of Pansorbin. The invisible pellet was resuspended in 250 μl of slurry without a wash step (Cont conc), and revealed a surprisingly high titre of 1.6×10⁶ cfu/ml; an enhancement of 115-fold compared to the control, though >60 fold less than that achieved with Pansorbin alone. Theoretically a recovery between 68-123% (depending on comparison either with T=0 or T=3 as the control) of retrovirus should not be possible under these conditions, as a 2600 g for 10 minutes is vastly less than any other centrifugation protocol both in terms of duration and g force.

In order to exclude the possibility that the extracellular Ig-like domains of B7-1³⁵ may be represented on the surface of the retrovirus^{36,37} and bind to the Pansorbin protein A, the concentration was performed in parallel using Sansorbin (protein A negative Staphylococcus aureus, Wood 46). The retrovirus could still be concentrated to 1.15×10⁷ cfu/ml using Sansorbin though this represents a 10-fold lower efficiency (also seen in Sansorbin without centrifugation) than that achieved with Pansorbin, but still better than centrifugation alone. This would indicate that the protein A component of the Pansorbin concentration may make a major contribution to the concentration effect. However, it is also possible that the Sansorbin (Wood 46) expresses protein A at a level that is normally undetectable, or that Wood 46 has a lower affinity for fibronectin.

Retroviral Concentration is not Target Cell Specific

To demonstrate that this effect was not specific to K562 cells alone, the same retroviral preparation was used in parallel to infect two other human myeloid cell lines, NB4 and U937, these are shown in FIG. 4 alongside a separate retroviral preparation used to infect human adherent epithelial HeLa cells. The titre of the control 3 hour incubation of the retroviral supernatant is extremely low on NB4 cells (46±40.4) with a huge standard deviation, this was determined using 100 μl of neat supernatant diluted in 1 ml of cell suspension. After a reduction in volume of 180 fold, the effective titre was increased to 1.3×10⁴ cfu/ml representing an increased titre of only 282 fold.

U937 cells did not infect at all using 100 μl of neat supernatant diluted in 1 ml of cell suspension, thus identical PG13 derived supernatant having a titre of 7.2×10³ cfu/ml on K562 cells (FIG. 3) is reduced by 150-fold on NB4 cells and to zero on U937 cells. Reliable infection of U937 cells is only achieved after concentration (P conc, 1.3×10³ cfu/ml) and since the initial titre was zero no effectiveness of concentration can be estimated. Finally, for Hela cells, the initial titre of retroviral supernatant of 410±46 is increased more than 4×10³-fold after a 160-fold concentration (P conc, 1.65×10⁶ cfu/ml). Although the initial titre of the virus (in the case of K562, NB4, and U937 the same viral preparation on the same day) can vary hugely, the titre is increased in all cases by incubation and centrifugation with Pansorbin, though not particularly effectively in the case of NB4.

Retroviral Concentration is not Vector insert Specific

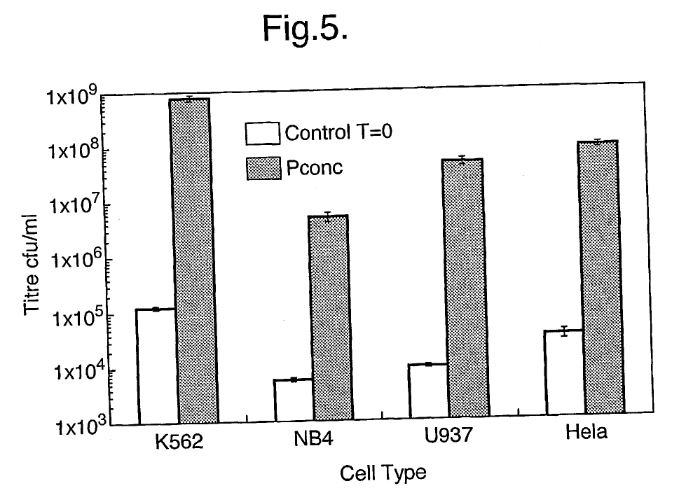

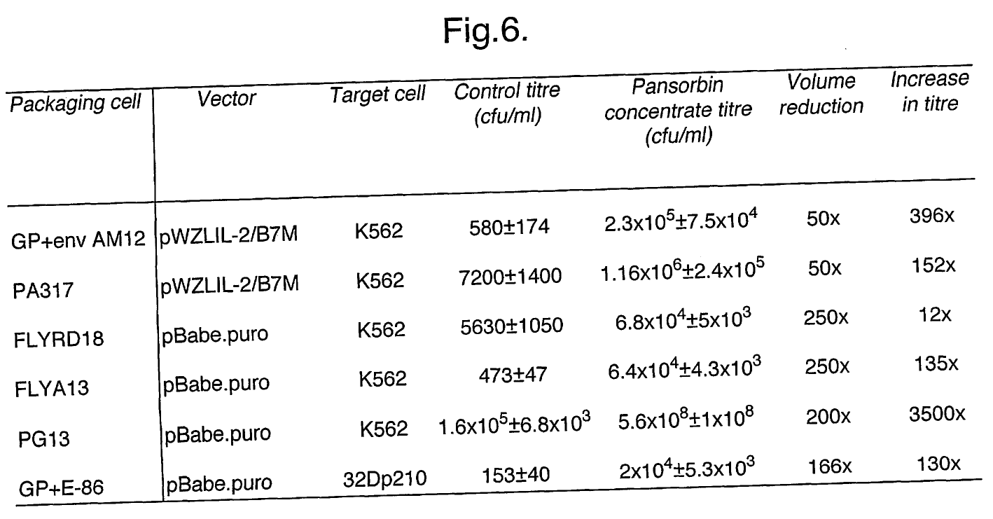

Having shown that concentration of retrovirus was effective (though variably) on other cell targets we sought to determine whether the retroviral conjugation with Pansorbin was an effect limited to a specific insert in the retroviral vector (ie B7-1), or applicable to other retroviral vectors shed from PG13. The inventors extended the study to investigate to same retroviral vector spine (pWZLblast³⁸) as the pWZLIL2/B7M Fusagene³⁰, but with an alternative insert. This vector, encodes a truncated gene with no signal peptide, and is referred to as RaRT (truncated retinoic acid receptor a). FIG. 5 shows that this mixed population of PG13 producer cells sheds retrovirus that can be concentrated in an identical fashion. Titration on K562 cells immediately after filtration showed a titre of 1.25×10⁵ cfu/ml (roughly 10 fold higher than IL-2/B7). Pansorbin mediated centrifugal concentration elevated this titre to 7.7×10⁸ cfu/ml (P conc, 6333-fold increase) after a 200-fold reduction in volume. The control retroviral concentration with centrifugation (and washing) demonstrated a 50-fold increase in titre (6.6×10⁶±8.6×10⁵ cfu/ml), for a reduction in volume of 200-fold (Data not plotted).

The same concentrated retroviral preparation again shows a limited (875 fold) increase in titre when used to infect NB4 cells (T=0: 5.6×10³ cfu/ml, P conc 4.9×10⁶ cfu/ml) cells. The starting titre of this viral supernatant on U937 in this case is 9.4×10³ cfu/ml which rose to 4.65×10⁷ cfu/ml in P conc (4900 fold), HeLa cells, with a starting titre of 3.35×10⁴ cfu/ml, show a less spectacular increase in titre of approximately 2500-fold.

Retroviral Concentration not Specific to PG13

Pansorbin mediated concentration works with different vectors shed from PG13 and is not specific to K562 target cells. A limited survey of other current packaging cell lines and different inserts is shown in Table 1. PG13 packaging cells appear to concentrate the most efficiently (3500 fold for 200 fold reduction in volume, in this case). However, this is closely followed by that of GP+envAM12³⁹ (1500 fold increase in titre when normalized for 200 fold concentration) and PA317⁴⁰(600 fold increase when normalized to 200 fold concentration). Murine fibroblast derived packaging cells seem to respond in a completely different manner to the human sarcoma cell derived FLY⁵ packaging cells for whom Pansorbin mediated concentration is largely ineffective. The GP+E-86⁴¹ cells being ecotrophic could only be tested on murine target cells, thus a comparison with those infecting human cells is not appropriate, but retrovirus from these cells do not concentrate very effectively.

Discussion

The use of Pansorbin as the insoluble dense substrate is a proof of principle that such particles supplied at high density would be able to interact with relatively low titre retroviral supernatant within the lifetime of infectious retrovirus. In FIG. 1 it is shown that the co-incubation of retroviral supernatant with Pansorbin can enhance the effective titre (in cfu/ml) of retrovirus. Examination of the time course of this interaction (FIG. 2) shows that although the initial interaction is quite rapid, best results were achieved after more that 60 minutes incubation. The inventors interpret these results as demonstrating that the retrovirus becomes complexed with the Pansorbin, a percentage of which can then settle under gravity in a static culture and increase the local concentration of retrovirus associated with the target cells. It is also apparent that the exposure of K562 cells to Pansorbin is not toxic, despite the overnight incubation. It could be argued that rather than promoting infection, the Pansorbin was chelating some inhibitory factor that may be in the supernatant^{8,22}. However, the inventors think this explanation unlikely as the addition of supernatant from non infected PG13 producer cells does not inhibit the effective titre of retrovirus in a standard infection protocol (data not shown), and filtration of retrovirus/Pansorbin coincubate indicated the cfu activity separating with the Pansorbin. It is also clear that retrovirus does not need to be dissociated from the Pansorbin for it to be infectious, or that overnight culture at 37° C. alone is enough to promote its release. However, this may not be the case in other procedures using Pansorbin and anti-retroviral envelope antibodies for mediating complex formation, in this case infectivity of the retrovirus appears to be compromised⁴².

Using data represented in FIG. 1 and 2 a rational choice of Pansorbin volume and co-incubation time was chosen and used both in scale up experiments and centrifugal concentration. Pansorbin/retroviral interactions are sufficiently strong in our protocol to allow two rounds of centrifugation at 2600 g, and relatively vigorous resuspension in fresh medium prior to titration. The combination of the reduced volume and the increased infectivity mediated by gravity in static culture may be one explanation for the fact that increases in titre assayed on K562 are far above that expected by reduced volume alone. It is also possible that some target cells may have an affinity for Pansorbin that can retain retrovirus in the vicinity of the target cell. FIG. 3 shows that, whatever the mechanism, the effective titre can be increase by up to 7500-fold for a volume reduction of only 180-fold. It was an additional surprise that centrifugation of the supernatant in the absence of Pansorbin was also capable of increasing the titre, although much less efficiently. This may indicate that PG13 derived retrovirus can become complexed with an unknown factor in culture resulting in crosslinking and the condensation of a centrifugable precipitate.

The depletion study in FIG. 3 (Pdep) was not clear; the inventors interpret the activity of the supernatant to indicate that at these speeds the Pansorbin is not fully pelleted. A clearer picture emerged in those samples where the complexed retrovirus was passed through a 0.45 μm filter, reducing the titre from that of the pre-filter by as much as 90% (P:P Filter). Once again however the titre did not drop to zero, which may indicate that much of the available retrovirus remains unbound to the Pansorbin (even with an optimized protocol) or that the filtration is sufficient to dissociate the bound retrovirus from the Pansorbin.

In the absence of a negative control for the concentration protocol (ideally Pansorbin with no retroviral binding activity) the activity of the protein A on the Pansorbin cannot be entirely discounted. Parallel experiments with Sansorbin show that the effect may be quite complex, although the Sansorbin is far less effective than its protein A positive counterpart it still gives quite a encouraging results. Sansorbin (Wood 46 strain) is not a genetically engineered substrain of Cowan I (Pansorbin) lacking only protein A, but a completely different strain, thus it may be that quite different cell surface proteins may also be lacking in these cells. Attempts to reproduce the concentration with streptavidin conjugated Paramagnetic beads complexed with a biotinylated protein A purified from a secreting variant S aureus strain showed only poor retroviral binding activity (data not shown), which may help to discount protein A as a ligand.

To determine how widespread the application of this concentration methodology may be, the inventors wished to determine whether K562 is a unique target cell for this type of procedure. FIG. 4 shows quite clearly that adherent HeLa cells respond in much the same way but to a lesser extent than K562 cells. The low titre of the IL-2/B7 retrovirus infectivity of U937 cells also meant that no estimate of the ratio of concentration could be made in these cells.

Although the ability of concentrated virus to infect cell lines was not cell specific it was important to determine whether the tropism of the retrovirus for the Pansorbin was unique to the IL-2/B7 insert. Therefore, the inventors repeated the protocol with other vectors, such as the RaRT insert in pWZLblast. Results detailed in FIG. 5 show that the ability to concentrate retroviral vectors is not specific to a given insert/vector construct. However, it is interesting to note that in this case the U937 target cells are more receptive to infection than the NB4 cells. This may be due an insert effect such as the expression of IL-2 being toxic to the U937 cells, although the inventors have been unable to show any inhibition in soft agar cloning efficiency in the three suspension cells with single additions of human IL-2 at concentrations as high as 50000 U/ml. The cloning efficiency of U937, K562 and NB4 was 78%, 43% and 10% respectively in the presence or absence of IL-2 (cells plated at 200 cells/dish; data not shown). Thus, externally applied IL-2 is not toxic to these cells, but regulated expression vectors will be required in order to determine if the presence of de novo intracellular IL-2 is differentially toxic to these cells.

Concentration by Pansorbin centrifugation is performed routinely on 50 ml samples of supernatant, and scaling up this protocol is primarily dependent on the amount of supernatant that can be input into the system. One can centrifuge 400 ml of supernatant every 10 minutes using 50 ml centrifuge tubes, making the production of virus from litres of supernatant relatively easy. This study was initially performed in response to a problem with low titres of virus and it is interesting to speculate on what could be achieved with high titre starting material. Pansorbin is purchased as a 10% (w/v) suspension, but an estimate of the number of particles can be obtained by haemocytometer counting and indicates that the concentration is between 1×10¹⁰ and 1×10¹¹/ml. Thus, the standard protocol for concentration of 50 ml of supernatant (250 μl Pansorbin) will use around 1.25×10¹⁰ particles, and even assuming that each particle can bind only one infectious retrovirus particle this gives a carrying capacity of Pansorbin of 1.25×10¹⁰ retroviral particles in 50 ml supernatant. It is probable that this method may be equally (or more) efficient with high titre producer cells than with the ones the inventors have studied. Assuming a supernatant contains 1×10⁶ cfu/ml of retrovirus, when mixed with the normal concentration of Pansorbin (2.5×10⁸/ml) each retrovirus would be an average of 100 μm from its neighbour and the Pansorbin particles only an average of 16 μm apart. Thus, the retrovirus need only travel 10-20 μm before arriving in the proximity of a Pansorbin particle, therefore increasing or reducing the retrovirus content of the supernatant (providing it is below the carrying capacity of the Pansorbin) would make little difference to the probability of the retrovirus contacting a Pansorbin particle. One might expect that the retrovirus would be unable to infect target cells when bound to Pansorbin, and indeed, it may be that when diluted with the target cells at 37° C. there is a back reaction releasing retrovirus gradually into the medium. However, the inventors believe that the retrovirus remains complexed to the Pansorbin whilst infecting the target cells.

Although, there is no definitive data regarding the mechanism for binding, the inventors are confident that the virus is bound to the Pansorbin, otherwise filtration of co-incubated supernatant would not reduce titre as seen in FIG. 3 (P and PFilter).

The first aspect of the present invention has been shown herein to work on PG13 cells for which the concentration procedure was optimized. However, using an identical protocol for targeting K562 cells, the inventors have also found that Pansorbin alone works well for GP+envAM12³⁹ and PA317⁴⁰ (mouse packaging cells, amphotropic murine leukemia virus envelope), with an increase in titre of 1500 and 600-fold, respectively, when normalized to a 200-fold reduction in volume. Further, the method has been carried out in other packaging cell lines such as FLYA13⁵ (human HT1080 fibrosarcoma cells, amphotropic murine leukemia virus envelope), FLYRD18⁵ (HT1080 with RD114 feline endogenous virus envelope). In addition the inventors have found that retrovirus from ecotropic packaging cells GP+E-86⁴¹ (Mo-MULV env) titred on murine 32Dp210⁴³ myeloid cells can be Pansorbin concentrated by 150-fold for a 200-fold reduction in volume, an efficiency in line with FLYA13 on K562 cells.

The inventors believe that binding of retrovirus to Pansorbin may be mediated by a fibronectin intermediary. The protein products of at least two bacterial genes appear to be specialized membrane bound fibronectin binding proteins (fnbA³² and fnbB³³), both of which were cloned from Staphylococcus aureus (though not Cowan I strain). However the fibronectin binding ability of Cowan I can be blocked by the exogenous application of short recombinant fnbB peptides³³ (fnbA not tested). Although the Wood 46 strain has not been directly tested in this way, it has been demonstrated that the ability of Cowan I to aggregate in the presence of either fibronectin or laminin is essentially absent in Wood 46⁴⁴. It is also of interest that both these strains adhere identically to fibronectin-coated tissue culture plasticware⁴⁴ indicating that Wood 46 may be deficient in either fnbA or fnbB but probably not both. It has also been reported that retrovirus derived from packaging cell lines GP+E-86, GP+envAM12, PA317 and PG13 all bind a recombinant fragment of human fibronectin (CH-296^{16,31}, otherwise known as RetroNectin™). Therefore, it is not unreasonable that retrovirus and fibronectin produced from NIH3T3 derived packaging cells⁴⁵ can become bound to fibronectin in the course of culture, and such fibronectin/retrovirus complexes may then bind Pansorbin via fnbA or fnbB. The level of expression of fibronectin from most packaging cells must be a limiting factor in the determination of retroviral titre otherwise no enhancement would be observed after RetroNectin treatment. It is thus of interest that, though the murine packaging cells have not been formally tested for the secretion of fibronectin, the human HT1080 fibrosarcoma cells at the core of FLYA13 and FLYRD18 express only 0.004% of total protein as fibronectin compared with the 0.3% associated with normal human diploid fibroblasts⁴⁶, and the FLYRD18 envelope may not even bind fibronectin⁴⁷. The FLYA13 cells, despite utilizing the'same env protein as both GP+envAM12 and PA317, also perform rather poorly in concentration assays.

Pansorbin/fibronectin/retrovirus complexes may also promote infection in a way analogous to RetroNectin, in that cell binding domains in fibronectin may crosslink to the target cells¹⁶, this may be less effective in NB4 cells since much of the VLA-4 and VLA-5 they express may be inactive⁴⁸.

The inventors have used four different batches of Pansorbin, two of Sansorbin and one batch of Pansorbin equivalent from Sigma Aldrich. Whilst they do observed batch variability of 2-4 fold in the concentrating ability of Pansorbin (2000-7500 fold increase in titre after concentration), they find it is always more efficient than Sansorbin, which is in turn always better than the Sigma Aldrich product. At first sight such batch variations may be considered a concern. However, the methods of preparation of Cowan I have been optimized and quality controlled on the basis of Protein A and not fnb. The poor activity of the Sigma Aldrich product appears to be related to how well it pellets at 2600 g, thus optimizing for better centrifugation may improve its activity.

Although the present invention has obvious applicability for in vitro studies, the applicability for therapy may be considered debatable as even in vitro transduced cells will have to spend time in the presence of high concentrations of Pansorbin. One may think that toxic factors may leach off Pansorbin in the course of infection, and these may be toxic in vivo if they become adhered to a cell vaccine. However, this possibility has been dismissed by other authors in the field. However, despite this, only protein A component of Pansorbin has been licensed for use in humans (management of autoimmune thromobocytopenia purpura, ITPP)⁵⁰. Further, following promising results in virus-induced rat malignancy⁵¹ extracorporeal adsorption of patient plasma using only protein A has reached the stage of clinical trials for metastatic breast cancer^{52,53}, Kaposi's sarcoma⁵³ and colon carcinoma⁵³ on the basis of removal of antibody complexes thought to inhibit anti-tumour immune responses^{52,53}. It is however tempting to speculate on the relative contributions of the Protein A and fnb on Cowan I in treatment of other malignancies where evidence for the role of retrovirus is more compelling and reduction in viremia would be dependent upon circulating retroviral/antibody complexes^{54,55}.

The effect of Pansorbin demonstrates that interactions between retrovirus and a particulate substrate can take place within the half-life of the retrovirus. This has allowed us to develop a simple procedure for the concentration of retroviral vectors. However, the further aspects of the invention are modifications which use reagents which may be considered more conducive to clinical practice and applicable to a wide range of packaging cell lines.

Materials and Methods Relating to the First Aspect of the Present Invention.

Cell Lines

Human (NB4, U937, K562) and mouse (32Dp210⁴³) myeloid cells lines and HeLa epithelial cells were grown routinely in RPMI+10% FCS, 2 mM L-glutamine, 100 μg/ml streptomycin and 100 U/ml penicillin (all Sigma, Poole, UK). Suspension cells were maintained between 1×10⁵ and 1>10⁶/ml whilst HeLa cells were passaged by trypsinization and maintained below 7×10⁶/90 mm tissue culture dish.

The mouse embryo fibroblast derived GP+envE-86⁴¹, PG13 GaLv pseudotype³ (CRL-10686, obtained from the ATCC), GP+envAM12, GP+E-86, PA317 and Human sarcoma derived FLYA13 and FLYRD18 packaging cells were routinely cultured in DMEM+10% FCS, 2 mM L-glutamine, 100 μg/ml streptomycin and 100 U/ml penicillin. Cells were maintained in 90 mm tissue culture dishes, passaged by trypsinization, and maintained between 5×10⁵ and 7×10⁶/90 mm tissue culture dish.

Reagents

Pansorbin (Calbiochem-Novabiochem, Nottingham, 507858) a 10% (w/v) suspension of heat-killed, formalin fixed, Staphylococcus aureus (Cowan I), 1 μm particles bearing a high cell-surface density of protein A was stored at 4° C., and replaced after 4-6 weeks. Sansorbin (Calbiochem-Novabiochem, Nottingham, 557601) a 10% (w/v) suspension of heat-killed, formalin fixed, Staphylococcus aureus (Wood 46), 1 μm particles with no cell-surface protein A was stored at 4° C., and replaced after 4-6 weeks. Insoluble protein A (Sigma, Poole, UK, P-7155) cell suspension approx 10% (wet weight/vol) of non-viable Cowan strain S. aureus, stored at 4° C., and replaced after 4-6 weeks. Protein A-biotin labeled (Sigma, Poole, UK P-2165) purified from culture medium of a protein A-secreting S. aureus strain (2 mg/ml in PBS pH 8.0, stored at −20° C.). Blasticidin S Hydrochloride (ICN Pharmaceuticals, Basingstoke, UK, 150477), stored filter sterile, −20° C. at 5 mg/ml in water. Puromycin (Sigma, Poole, UK, P-8833) stored filter sterile, −20° C. at 5 mg/ml in water. Agar Noble (Difco Laboratories, Detroit, USA, 0142-17-0). Polybrene (Sigma, Poole, UK, H-9268) made up in water to 8 mg/ml and stored filter sterile at −20° C. Streptavidin MagneSpheres Paramagnetic particles (Promega, Madison, USA, Z5482) 1 mg/ml (5×10⁸ particles/ml) of 1 μm diameter paramagnetic particles in PBS stored at 4° C.

Generation and Culture of Producer Cells

PG13, GP+envAM12 and PA317 packaging cells were trypsinized and plated at 1×10⁶/90 mm dish; after 4 hours the medium was aspirated and replaced with 10 ml of filtered (0.45 mM) GP+E-86 supernatant containing 4 μg/ml polybrene. These calcium phosphate transfected GP+E-86 mixed cell populations produced pWZLIL-2/B7³⁰ or pWZLRaRT retroviral vectors (conferring resistance to Blasticidin S) or pBabe.puro vectors (conferring resistance to Puromycin). This infection was repeated after 24 hours, the cells cultured for a further 48 hours and the cells selected in DMEM+10% FCS containing 10 μg/ml Blasticidin S or 5 μg/ml Puromycin as appropriate for four weeks and a mixed population of resistant cells cryopreserved.

FLYRD18 and FLYA13 packaging cells were initiated at 7.5×10⁵/90 mm dish, after overnight culture the medium was aspirated and replaced with 10 ml of filtered (0.45 μm) PG13.pBabe.puro supernatant containing 8 μg/ml polybrene. After 72 hours the medium was aspirated and replaced with fresh medium containing 5 μg/ml Puromycin. Cells were culture for a further 7 days at which point all control cultures were dead and the mixed population of survivors cryopreserved.

Generation of Retrovirus

PG13, GP+envAM12, PA317 and GP+E-86 producer cells were trypsinized and plated at 1×10⁶/90 mm dish, after 72 hours the medium was replaced; 24 hours later the medium was aspirated and filtered through a 0.45 μm filter and taken for further processing. FLYA13 and FLYRD18 cells plated at 2×10⁶/90 mm dish, after 48 and 72 hours the medium was replaced, and after a total of 96 hours later the medium was aspirated and filtered through a 0.45 μm filter and taken for further processing.

Preparation of Staphylococcus aureus.

Pansorbin, Sansorbin or insoluble protein A was diluted 1:20 in RPMI+10% FCS and stored at 4° C. for 18 hours. The required amount was centrifuged (2600 g, 20 minutes, 4° C.) and resuspended to the desired concentration in RPMI+10% FCS.

Preparation and Concentration of Pansorbin/Sansorbin Retrovirus Complexes

The indicated volumes of Pansorbin/Sansorbin were added to the desired volume of retroviral supernatant in sterile polypropylene tubes and the mix incubated at 4° C. under constant motion (Stuart Scientific SRT1 tilting roller mixer). At the indicated times the mix was either taken directly for the determination of cfu/ml titre or concentrated by centrifugation. For concentration: 45 or 50 ml of mix were centrifuged (2600 g, 10 minutes, 4° C.), the supernatant discarded, the pellet resuspended in 10 ml of cold DMEM+10% FCS and centrifuged once again as above. The supernatant was poured off and the tubes stored inverted for 60 seconds to drain and the pellet resuspended in approximately 250 μl of cold RPMI+10% FCS. Resuspending the pellet can be difficult as it tends to clump (especially Pansorbin), and 250 μl final volume tends to be composed of 50% packed volume and slurry and 50% fresh medium. If necessary the mixture can be pulsed at 150 g (to allow efficient recovery) and removed to polypropylene cryovials for storage on ice and determination of cfu/ml titre.

Determination of cfu/ml Titre

Suspensions of K562, U937, NB4 and 32Dp210 cells were counted and adjusted to 4×10⁵/ml in RPMI+10% FCS with polybrene at 4.4 μg/ml. The cells were then plated in 24 well cell culture plates in aliquots of 1 ml and incubated at 37° C./5% CO₂ for 1-3 hours. Retroviral preparations were serially diluted 1:10 in RPMI+10% FCS, 10 μl added to triplicate wells and mixed thoroughly. After 18-24 0.9 ml of cells was mixed with 3.8 ml of RPMI+24% FCS+1.3 mM sodium pyruvate and maintained at 37° C. followed by an additional 0.3 ml of autoclaved 5% w/v Noble Agar in water (final concentration 0.3%) which had been maintained at 60° C. The cells were then plated in 60 mm tissue culture dishes and, after allowing the agar to set, placed at 37° C./5% CO₂. After a further 18-24 hours an additional 5 ml of RPMI+20% FCS+1 mM sodium pyruvate containing either 20 μg/ml Blasticidin S or 10 μg/ml Puromycin was carefully added, resulting in a soft agar selection concentration of 10 and 5 μg/ml respectively. The dishes were returned to culture for a further 2-3 weeks after which soft agar colony number was determined. The concentration in cfu/ml was calculated as the number of colonies per dish/well multiplied by the dilution factor. In all cfu/ml determinations multiple dilutions were initiated although most would be non-informative, having either too few colonies, or too many to count accurately (ie more than 300/60 mm dish).

Adherent HeLa cells were trypsinized, counted and adjusted 1×10⁵/ml in RPMI+10% FCS and then plated in 24 well cell culture plates in aliquots of 0.5 ml. After 18 hours incubation (37° C./5% CO₂) and 1-3 hours prior to infection an additional 0.5 ml of medium containing polybrene was added to bring the final concentration to 4.4 μg/ml.

Retrovirus was subjected to serial 1:10 dilution's in RPMI+10% FCS and triplicate 100 μl aliquots of the appropriate dilution added to, and mixed with, the target cells. After 48 hours infection, the medium was replaced with fresh medium containing 10 μg/ml Blasticidin S. The medium was replaced every 3-4 days for 2 weeks after which it was aspirated, the plates stained with 2 ml Commasie blue stain (5.35% wt/vol in 45% methanol: 10% acetic acid), washed in tap water, colonies counted, and the titre determined.

### DETAILED DESCRIPTION RELATING TO THE SECOND, THIRD AND FOURTH ASPECTS OF THE PRESENT INVENTION

Paramagnetic Particle Mediated Concentration of Fibronectin Associated PG13 Derived Retrovirus.

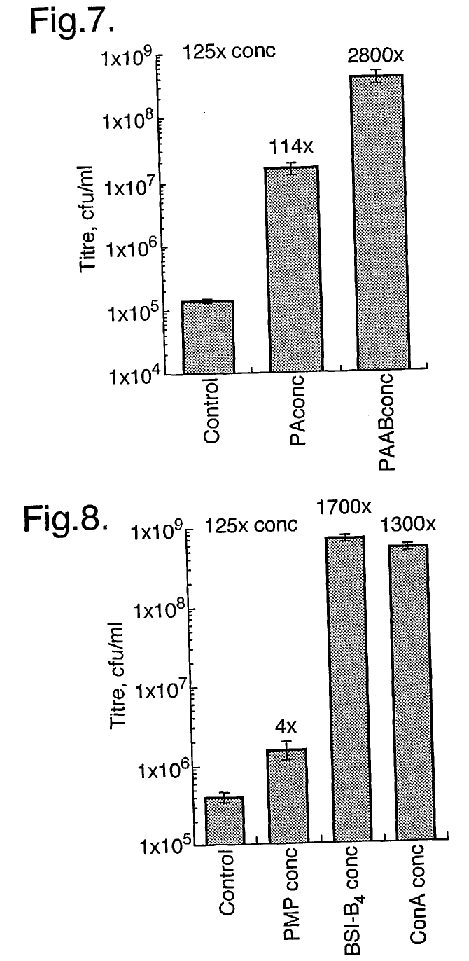

In order to determine whether PG13 derived retrovirus is indeed associated with fibronectin in packaging cell supernatant the present inventors used a polyclonal antibody directed against murine fibronectin coupled to PMPs. The ability of these particles to capture infectious PG13 retrovirus as assayed using the previously described soft agar colony formation assay⁵⁶ are detailed in FIG. 7. In the absence of concentration the initial titre (cfu/ml) on human myeloid K562 cells is 1.4×10⁵±9×10³ cfu/ml (control), concentration of this with protein A-biotin conjugated PMPs (PAB) by a 125 fold reduction in supernatant volume increased this titre by more then 100 fold to 1.6×10⁷±3.4×10⁶ cfu/ml. However, polyclonal rabbit anti mouse fibronectin, antibodies conjugated to the PMPs via protein A-biotin (protein A antibody biotin :PAAB) increases the titre to 4×10⁸±9.5×10⁷ cfu/ml, representing an increase of 2800 fold for the same 125 fold volume reduction. Combinations of antibody protein A-biotin resulting in an orientation specific antibody anchorage is 25 times more efficient that protein A-biotin alone. However, the efficiency of protein A-biotin alone does not suggest a limited affinity of protein A for either fibronectin or retrovirus.

Antibodies to murine fibronectin allow infectious retrovirus from murine fibroblast derived packaging cells to be captured from retroviral supernatants. Volume reductions of only 125 fold result in increased titres of the retrovirus in the order of 2000-3000 fold. The total infectivity (cfu/ml) in a captured retrovirus population is roughly 20 times that of the control supernatant. This may reflect increased delivery of virus to cells by gravity mediated settling of PMPs, rather than brownian motion alone¹⁵, or increased infectivity of retrovirus after PMP capture. Depletion studies, using the supernatant from the retrovirus/PMP mixes after magnetic concentration, also show reductions of some 90% of the retroviral titre from the supernatant. This data confirms that PG13 packaging cells secrete murine fibronectin into the supernatant and that either most of the retroviral particles are associated with it, or, retrovirus in the absence of fibronectin is not infectious.

The efficiency of this method shows that under these conditions both the epitopes responsible for binding of retrovirus to fibronectin, and fibronectin to target cells are not sterically hindered by the antibody. This protocol uses a total of 1.25×10⁹ PMPs to concentrate the 5 mls of retroviral supernatant, additional experiments with reduced numbers of PMPs resulted in extensive cross-linking as evidenced by the clumping of PMPs. Other attempts using lower concentrations of protein A-biotin and antibody also resulted in lower efficiencies of retroviral concentration.

Lectin/PMP Mediated Retroviral Particle Capture

Retroviral particles shed from murine packaging cells are very sensitive to inactivation by human serum and this appears to be dependent on both the packaging cell and the envelope protein utilized⁵⁷. The majority of this sensitivity results from terminal Gal(α1-3)Gal modifications to the envelope proteins of retroviral particles shed by murine packaging cells. The human homologue of this murine enzyme possesses ancestrally acquired mutations that result in little (α1-3) galactosyltransferase activity being evident in human cells^{58,60}. The high levels of antibodies directed against Gal(α1-3)Gal modifications found in human serum ensure that retroviral vectors derived from murine packaging cells are rapidly inactivated in vivo⁵⁷ The presence of Gal(α1-3)Gal modifications on PG13 derived retrovirus would offer a second strategy for the capture of PG13 retrovirus. FIG. 8 shows the efficiency of concentration using PMPs conjugated with either the Gal(α1-3)Gal binding Isolectin B4 (BSI-B4)⁶¹ or ConcanavalinA (ConA) which primarily binds more ubiquitous α-mannose modifications⁶². Using either of these two lectins conjugated with PMPs a control titre of 4×10⁵±5.5×10⁴ cfu/ml can be elevated to 7×10⁸±6.4×10⁷ cfu/ml (1700 fold) with biotin-BSI-B₄ or 5.4×10⁸±5×10⁷ cfu/ml (1300 fold) with biotin-ConA, after a 125 fold reduction in supernatant volume. The concentration by PMPs alone resulted in a4 fold increase (1.6×10⁶±4×10⁵ cfu/ml) demonstrating that retroviral concentration was dependent on lectin, rather than streptavidin. This strategy may be more broadly applicable as it may be effective for retroviral vectors unable to bind fibronectin⁴⁷. It is surprising in this case that the retrovirus remains infectious since it is closely associated with the PMP and not captured via a large intermediate protein like fibronectin as in the second aspect of the present invention. This is especially true for ConA, which is one lectin known to inhibit in vitro infection by HIV-1⁶³, though this may be a special case reflecting its unique receptor usage⁶⁴. It is also interesting that a similar ConA capture strategy virtually eliminated the infective titre of HIV-1 (reduced by more than 95%)⁶⁵, a marked contrast to the increase seen with PG13 derived vectors. It is, however, also possible that lectins bind proteins in the supernatant that act as intermediaries, in a manner analogous to fibronectin, and thus not directly to the retrovirus itself.

Lectin/PMP Mediated Magnetic Concentration Applied to Human HT1080 Derived Packaging Cells.

The lectin strategy has also been tested on FLYA13 and FLYRD18 derived retrovirus^{5,66}, for which the inventors had previously been unable to design an effective method.. FIG. 9 shows the lectin/PMP mediated concentration of FLYRD18 and FLYA13 derived retrovirus. FLYRD18 cells have a (Control) titre of 3.5×10⁴±1.1×10⁴ cfu/ml, ConA mediated concentration (125 fold) increased this to 1.2×10⁷±3.6×10⁶ cfu/ml. A342 fold increase such as this is far short of the 1300 fold achievable with PG13 and indicates ConA/PMP mediated concentration of FLYRD18 retrovirus is routinely five times less efficient than for those derived from PG13. The titre of the BSI-B₄/PMP concentrate is only 6 times higher (2.2×10⁵±6.4×10⁴ cfu/ml) than the Control, and confirms the minimal presence of α-Galactosyl groups on human cells. FLYA13 cells have a (Control) titre of 6.2×10±5.7×10² cfu/ml, which increases 369 fold to 2.3×10⁶±2×10⁵ cfu/ml after a ConA/PMP mediated reduction in volume of 125 fold. Once again the BSI-B₄ was ineffective at capturing retrovirus shed from human derived packaging cells. Despite being less effective for FLY than PG13 derived retrovirus, the lectin mediated concentration is still the most efficient yet described for the family of HT1080 (FLY) derived retrovirus⁵.

Biotin Succinimide Ester/PMP Mediated PG13 Retroviral Particle Capture

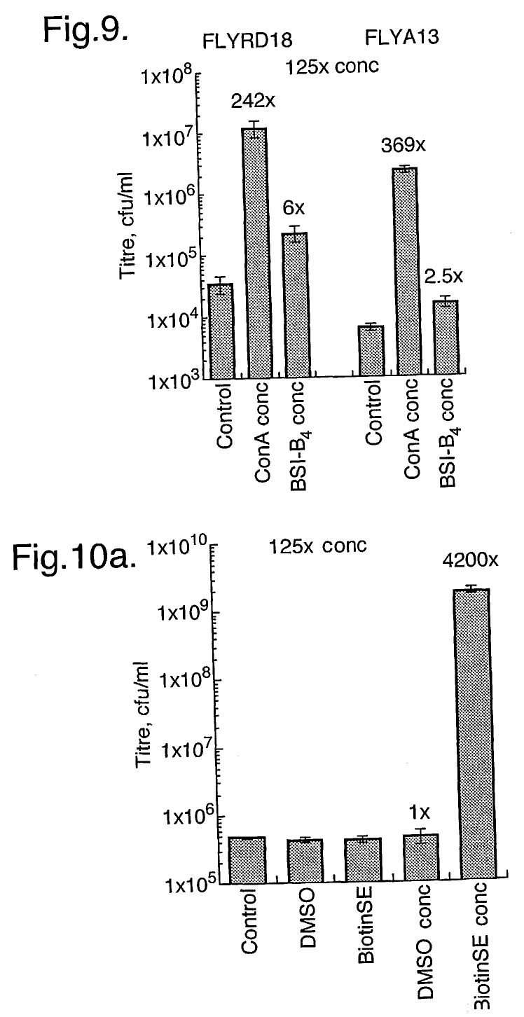

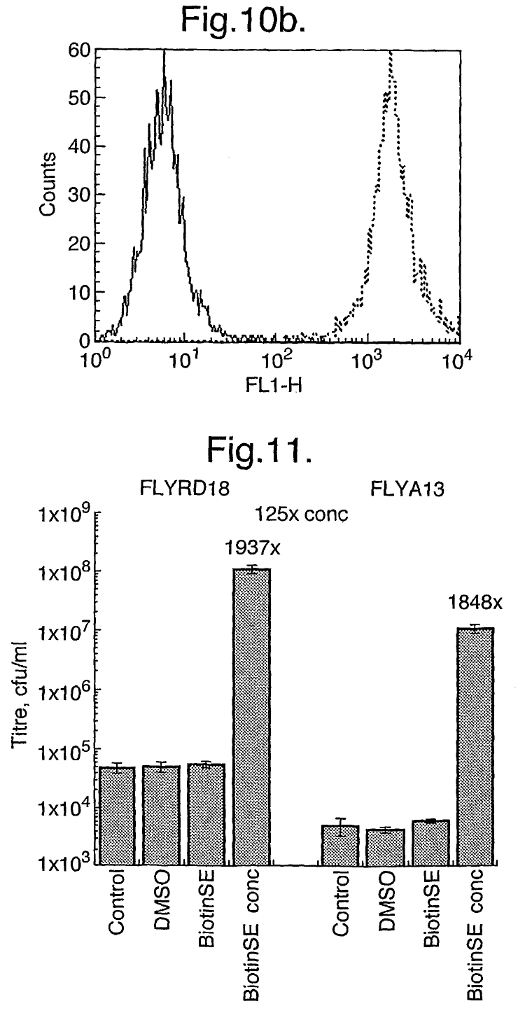

Both the second aspect of the present invention (antibody mediated) and the third aspect of the present invention (lectin mediated) may not be universally applicable or efficient for all enveloped retroviral packaging cell lines used in gene therapy to date. Thus, the inventors have designed a fourth and perhaps most powerful aspect of the invention for the PMP mediated concentration of retrovirus. Using the methodology for labeling packaging cells^{67,68}, PG13 packaging cells were labelled with a succinimide ester derivative that covalently couple biotin to proteins on the surface of packaging cells. FIG. 10a shows the results of PMP mediated concentration of retrovirus derived from biotinylated packaging cells. In the absence of concentration the biotinylation (BSE) or carrier alone (DMSO) treatment of packaging cells has no effect on titre (Control: 4.6×10⁵±1.5×10⁴ cfu/ml, DMSO: 4.2×10⁵±2.6×10⁴ cfu/ml, BSE: 4.2×10⁵±3.6×10⁴ cfu/ml). In the absence of biotinylation (treatment of cells with DMSO carrier alone), the PMPs alone are incapable of capturing retrovirus (DMSO conc: 4.6×10⁵±1×10⁵ cfu/ml). Biotinylated packaging cells secrete a retrovirus that affinity couples to the streptavidin on the PMPs and allows concentration to a titre of 1.8×10⁹±1.8×10⁸ cfu/ml (4200 fold increase) after an only 125 fold reduction in volume appears to be the most effective strategy yet for PG13. Furthermore the titre of the depleted supernatant shows a reduction to 6.4×10⁴/ml±1.4×10⁴/ml representing a depletion of more than 80% compared to the control titre. FIG. 10b shows a FACS profile illustrating the extent of the surface modification detectable by avidin-FITC 24 hours after the labeling. Detailed on the profile is the fluorescence of control cells treated with DMSO alone and stained with Avidin-FITC (DMSO+Av-FITC) compared to that of cells treated with Biotin N-Hydroxysuccinimide ester 24 hours previously and stained with Avidin-FITC (BSE+Av-FITC). Even after 24 hours in culture there is a greater than 2 log shift in the fluorescence. The biotinylation reaction is not toxic to the packaging cells and is thought to target NH₂ termini on the side chains of the amino acid lysine. Previous experience with the biotinylation of suspension cells suggests that the surface modifications are quite rapidly endocytosed as a result of protein turnover. Therefore, little biotinylated protein is secreted into the supernatant. Envelope protein residing on the surface of the packaging cells may be biotinylated prior to is association with the retroviral gag and pol during the budding process. In this way biotin labeled retrovirus can be secreted from such a biotinylated packaging cell. Since other cell's surface proteins may also be represented as their biotinylated derivatives on the retrovirus coat, the env protein may not have to be biotinylated. In addition it is possible that packaging cell derived proteins that later become associated with the retrovirus have been the primary target for the biotinylation. There are estimated to be 300 envelope molecules on the surface of a retrovirus, thus the efficiency of the biotinylation process is not crucial to the capture as the strength of biotin/streptavidin interactions are so strong that only one biotin contact with the PMP may be sufficient to capture retrovirus.

Efficient Biotin Succinimide Ester/PMP Mediated Capture of FLYRD18 and A13.

Concanavalin A mediated capture of FLYRD18 and FLYA13 was not as successful as anticipated. Therefore the present inventors chose to investigate whether BiotinSE capture efficiently captured retrovirus from these packaging cells. FIG. 11 shows the result of experiments to determine the efficiency of biotin mediated magnetic concentration on FLYRD18 (FIG. 11) and FLYA13 (FIG. 11). The effect of carrier alone and biotin modification on starting titre was again examined, for FLYRD18 (FIG. 11) the Control titre (4.6×10⁴±8.7×10³ cfu/ml) was no different from that of DMSO carrier (4.7×10⁴±1.2×10⁴ cfu/ml) or the biotin modification (5.4×10⁴±3×10³ cfu/ml). Carrier (DMSO) alone treatment of cells did not result in concentration of the retrovirus after a 125 fold reduction in volume, DMSO conc: 4.3×10⁴±7×10³ cfu/ml. Biotinylation of FLYRD18 resulted in a concentrate titre of 1×10⁸±1.8×10⁷ cfu/ml, over 2000 fold greater than the starting material after a 125 fold reduction in volume, an efficiency approaching that of the PG13. It has also been determined that FLYA13 cells are also efficiently concentrated by this fourth aspect of the invention.

Freeze/Thaw Stability of Control and Concentrates PG13 Derived Retrovirus.

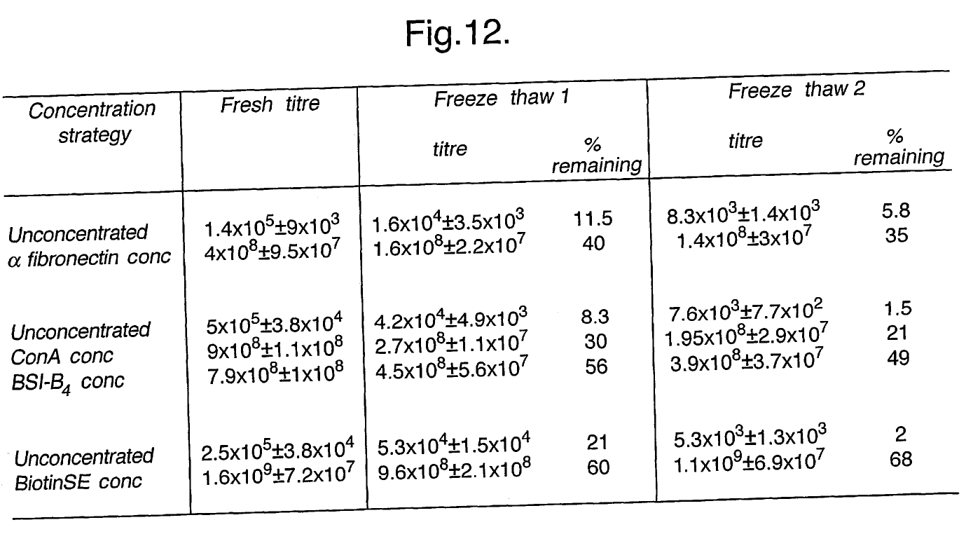

Retroviral preparations must be prepared in advance in order for the required safety testing to be independently undertaken, thus it was important to determine what implications the concentration had for the maintenance of infectivity after freezing. FIG. 12 shows that the retroviral concentrates are actually more stable than frozen control supernatants. The controls each lose more than 80% of their titre after the first freeze thaw, and loose more than 94% after the second. In the case of the concentrates, most of the activity is lost after the first thaw (up to 60%), but little additional infectivity is subsequently lost. Thus PG13 derived concentrates can be stored more efficiently than neat supernatant (in the case of the ConA and BS-IB₄after more than 6 weeks at −20° C.). As described below, the concentrate was frozen in a final concentration of 7.5% DMSO achieved by a three volume addition of 10% DMSO, 20% FCS (titres of thawed concentrates have therefore been adjusted to take account of this dilution). Since large dilutions are required in order to titrate the concentrate the effect of DMSO is not an issue, it is also possible to use the PMPs magnetic concentrator to further wash the retroviral preparation after thawing. However, freezing of control supernatant is not amenable to freezing in the presence of DMSO since even diluting the thawed retrovirus 10 fold would still contain significant DMSO contamination.

PMP/Retroviral Conjugates can be used for in vitro Localization of Infection.

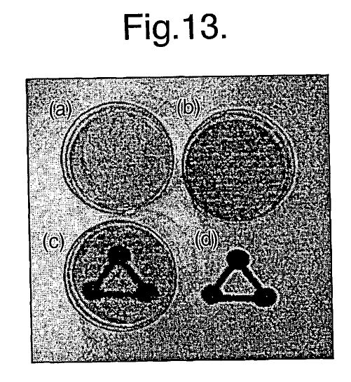

It is now possible to produce what are in effect “infectious, paramagnetic, retroviral vector particles” and these can then be magnetically attracted to the desired location for infection. FIG. 13 shows an example of in vitro magnetic localization and represents a proof of principle for this methodology. The illustrated shape (d), cut from magnetic sheeting, placed underneath a sub confluent culture of HeLa cells, can both attract and retain the retrovirus to primarily infect the area dictated solely by the presence of the magnet (c). The particular design used here is intended to show that this targeting is not the result of a fluid dynamic causing retroviral vectors to vortex in the dish during agitation. In addition this experiment shows that infection can take place in the continued presence of a magnetic field, and that the retrovirus remains captured by paramagnetic particles when in culture. This magnetic sheeting, though weak, is extremely effective in directing retroviral infection in vitro, however efficient in vivo targeting is a three dimensional problem and would require more intense magnetic fields. For in vivo environments, small permanent magnets could be used at accessible sites, and ex vivo generated electromagnetic fields could focus at one particular site at a time. As with all targeting, the inhibition of infection of those sites not targeted is a major problem, for this reason reversible inactivation of retroviral vectors such as those discussed are an exciting and complimentary development (28). Localization of reversibly inactivated retroviral vectors to specific tissues, organs and metastases followed by localized reactivation of infection may add a further level of sophistication to in vivo targeting.

In summary, streptavidin PMP technology coupled with the correct choice of ligand captures retrovirus derived from both PG13 and FLY packaging cells. This study examines only three packaging cell types and two target cell lines, it is therefore probable that additional optimization for each target cell type will further increase the infectivity of the concentrateas there are likely to be preferred capture methods dependent on both target cell type and receptor usage by the retrovirus. Concanavalin A, for instance, would not be the best choice for retrovirus entering via a-mannose modified receptors (25-27). Spare biotin binding capacity on PMPs may also be used to conjugate further proteins, introducing the kind of targeting so far only achievable by ligand (31-34), or single chain Fv modifications of retroviral envelope gene (35-38). The inventors realise that coupling ligands to an infectious formulation without individual genetic modifications will introduce greater flexibility in retroviral targeting and may negate the requirement for both pseudotyping (31, 33, 37), and target cell specific packaging cell design (32, 34, 35, 38). It is also worth noting that paramagnetic retroviral vectors in the absence of additional ligands represent a potential strategy for the in vivo targeting of infection to small groups of cells within a background of otherwise identical cells.

PMP/retroviral technology with modifications to optimize retrovirus/PMP ratio, reduce polycation enhancer dependence (39) and capture current lentiviral vector constructs (40) raises the possibility of retroviral infection in vivo applied as a PMP concentrate and either retained at or directed to the requires sites by magnetic fields.

Materials and Methods

Cell Lines

Human myeloid suspension (K562) and HeLa adherent epithelial cells were grown routinely in RPMI+10% FCS, 2 mM L-glutamine, 100 mg/ml streptomycin and 100 U/ml penicillin (all Sigma, Poole, UK). Suspension cells were maintained between 1×10⁵ and 1×10⁶/ml whilst HeLa cells were passaged by trypsinization and maintained below 7×10⁶/90 mm tissue culture dish.

PG13 GaLv pseudotype packaging cells³ (CRL-10686, obtained from the ATCC, Rockville, Md., USA) were routinely cultured in DMEM+10% FCS, 2 mM L-glutamine, 100 μg/ml streptomycin and 100 U/ml penicillin. Cells were maintained in 90 mm tissue culture dishes, passaged by trypsinization, and maintained between 5×10⁵ and 7×10⁶/90 mm tissue culture dish.

Reagents

Streptavidin MagneSpheres Paramagnetic particles, 1 mg/ml (5×10⁸ particles/ml) of 1 μm diameter paramagnetic particles in PBS stored at 4° C. Polyclonal I Rabbit anti mouse fibronectin, 10.8 mg/ml in PBS, stored at 4° C. (Biogenesis, Poole, UK, 4470-4339). Protein A-biotin labelled (Sigma, Poole, UK, P-2165) purified from culture medium of a protein A-secreting S. aureus strain (2 mg/ml in PBS pH 8.0, stored at −20° C.). Succinyl-Concanavalin A, biotin labeled (Sigma, Poole, UK, L0767) stored −20° C. at 1 mg/ml in PBS pH 8.0. Isolectin B₄ from Bandeiraea Simplicifolia BS-I biotin labelled (BS-IB₄), stored −20° C. at 1 mg/ml in PBS pH 8.0 (Sigma, Poole, UK, L2140). Biotinamidocaproate N-Hydroxysuccinimide ester) (Sigma, Poole, UK, B2643) reconstituted to 366 mM in DMSO and stored at −20° C. and henceforth referred to as BSE. Avidin-FITC (Sigma, Poole, UK, A-2901) stored at −20° C. reconstituted to 1 mg/ml in PBS pH 8.0. Puromycin (Sigma, Poole, UK, P-8833) stored filter sterile, −20° C. at 5 mg/ml in water.

Generation and Culture of Producer Cells

PG13 packaging cells were trypsinized and plated at 1×10⁶/90 mm dish; after 4 hours the medium was aspirated and replaced with 10 ml of filtered (0.45 μm) GP+E-86 supernatant containing 4 pg/ml polybrene. These calcium phosphate transfected GP+E-86 mixed cell populations produced pBabe.puro vectors conferring resistance to Puromycin. This infection was repeated after 24 hours, the cells cultured for a further 48 hours and the cells selected in DMEM+10% FCS containing 5 μg/ml Puromycin for four weeks and a mixed population of resistant cells cryopreserved.

FLYRD18 and FLYA13 packaging cells were initiated at 7.5×10⁵/90 mm dish after overnight culture the medium was aspirated and replaced with 10 ml of filtered (0.45 μm) PG13.pBabe.puro supernatant containing 8 μg/ml polybrene. After 72 hours the medium was aspirated and replaced with fresh medium containing 5 mg/ml Puromycin. Cells were cultured for a further 7 days at which point all control cultures were dead and the mixed population of survivors could be cryopreserved.

Generation of Retrovirus

PG13 producer cells were trypsinized and plated at 1×10⁶/90 mm dish, after 72 hours the medium was replaced; 24 hours later the medium was aspirated and filtered through a 0.45 μm filter and taken for further processing. FLYRD18 and FLYA13 were plated at 2×10⁶/90 mm dish, after 48 and 72 hours the medium was aspirated and replaced with fresh. After a further 24 hours the medium containing disabled retrovirus was aspirated and filtered through a 0.45 μm filter and taken for further processing.

Generation of Biotin Labeled Retrovirus

Cells were biotinylated essentially as described above with modifications for adherent cells^{67,68}. Briefly, after 72 hours culture the medium was thoroughly aspirated and washed with PBS pH 8.0 with additional 0.75 mM CaCl₂ and 0.48 mM MgCl₂, and replaced with 10 ml of freshly diluted BiotinSE (500 μM in PBS pH 8.0+Ca²+, Mg²+). Cells were incubated at room temperature for 30 minutes after which the reagent was thoroughly aspirated, replaced with fresh growth medium and the cells returned to culture at 3° C. After a further 3-4 hours the medium was again changed for fresh and the cells returned to culture for 18 hours after which retroviral supernatant was aspirated. In all cases control labeling with carrier alone (DMSO) were processed in parallel.

Immunofluorescence

Biotinylated packaging cells were cultured overnight. After harvesting retrovirus, cells were removed from the substratum with gentle pipetting in the presence of versene and placed in DMEM+10% FCS. A total of 1×10⁶ cells were labeled in 100 μl of HBSS+1% FCS with or without 10 μg/ml Avidin-FITC. Cells were incubation at room temperature and washed and analysed by flow cytometry.

Preparation of Paramagnetic Particles

For 5 ml of retroviral supernatant 2.5 ml of Paramagentic particles (PMP) (5×10⁸/ml) were placed in 15 ml polypropylene tubes and applied to a Dynal MPC-6 Magentic Particle Concentrator, the supernatant was aspirated and the PMPs resuspended in 1 ml of filter sterile PBS+0.1% BSA and transferred to a sterile 1.5 ml eppendorf tube. The PMPs were then applied to a Dynal MPC-E Magentic Particle Concentrator, and the supernatant aspirated and the PMPs resuspended in 0.4 ml filter sterile PBS+0.1% BSA and applied to the MPC-E, and the supernatant aspirated.

For antibody conjugation-PMPs were resuspended in 50 μl of PBS+0.1% BSA and 50 μl of 2 mg/ml ProteinA-biotin. After 30 minutes incubation at room temperature the PMPs were washed three times in PBS+0.1% BSA using the MPC-E and resuspended in 250 ml of 5 mg/ml Polyclonal Ig Rabbit anti mouse fibronectin in PBS+0.1% BSA.

For Lectin conjugation: PMPs were resuspended in either 100 μl of 1 mg/ml biotin Succinyl-Concanavalin A, 10 μl of 500 μg/ml biotin labelled Isolectin B₄ (BS-IB₄). For Biotin succinimide ester concentration and Control unconjugated PMPs-PMPs were resuspended in 100 μl of PBS+0.1% BSA.

After 30 minutes incubation at room temperature (with periodic agitation) all PMP conjugations were washed three times in 500 μl of PBS+0.1% BSA using the MPC-E. After the final wash the PMPs were resuspended in the desired volume of retroviral supernatant.

Preparation and Concentration of PMP: Retrovirus Complexes

Routinely 1.25×10⁹ prepared PMPs are resuspended in 5 ml of retroviral supernatant in sterile polypropylene tubes and the mix incubated at 4° C. under constant motion (Stuart Scientific SRT1 tilting roller mixer). After 2.5 hours the mix was applied to the Dynal MPC-6, the supernatant aspirated and the PMPs resuspended in 1 ml of either PBS+0.1% BSA or RPMI+10% FCS (Lectin concentration). The PMPs were then washed a further 3 times using the MPC-E, and the PMPs resuspended in the minimum volume of RPMI+10% FCS. A packed volume from 1.25×10⁹ PMPs can be resuspended in 20 μl of RPMI+10% FCS giving a final volume of 40 μl (5 ml: 40 μl, 125 fold volume) which is then further processed.

Determination of cfu/ml Titre

Suspensions of K562 cells were counted and adjusted to 4×10⁵/ml in RPMI+10% FCS with polybrene at 4.4 μg/ml. The cells were then plated in 24 well cell culture plates in aliquotes of 1 ml and incubated at 37° C./5% CO₂ for 1-3 hours. Retroviral preparations were serially diluted 1:10 in RPMI+10% FCS, 100 μl added to triplicate wells and mixed thoroughly. After 18-24 0.9 ml of cells was mixed with 3.8 ml of RPMI+24% FCS+1.3 mM sodium pyruvate and maintained at 37° C. followed by an additional 0.3 ml of autoclaved 5% Noble Agar (final concentration 0.3%) which had been maintained at 60° C. The cells were then plated in 60 mm tissue culture dishes and, after allowing the agar to set, placed at 37° C./5% CO₂. After a further 48 hours an additional 5 ml of RPMI+20% FCS+1 mM sodium pyruvate (containing either 10 μg/ml Puromycin) was carefully added, resulting in a soft agar selection concentration of 5 μg/ml Puromycin. The dishes were returned to culture for a further 2-3 weeks after which soft agar colony number was determined. The concentration in cfu/ml was calculated as the number of colonies per dish/well multiplied by the dilution factor. In all cfu/ml determinations multiple dilutions were initiated although most would be non-informative, having either too few colonies, or too many to count accurately (i.e. more than 300/60 mm dish).

Adherent HeLa cells were trypsinized, counted and adjusted 1×10⁵/ml (RPMI+10% FCS or DMEM+10% FCS as required) and then plated in 24 well cell culture plates in aliquots of 0.5 ml. After 18 hours incubation (37° C./5% CO₂) and 1-3 hours prior to infection an additional 0.5 ml of medium containing polybrene was added to bring the final concentration to 4.4 mg/ml.

Retrovirus was subjected to serial 1:10 dilution's in RPMI+10% FCS and triplicate 100 μl aliquots of the appropriate dilution added to, and mixed with, the target cells. After 48 hours infection, the medium was replaced with fresh medium containing 5 μg/ml Puromycin. The medium was replaced every 3-4 days for 2 weeks after which it was aspirated, the plates stained with 2 ml Commasie blue stain (5.35% wt/vol in 45% methanol: 10% acetic acid), washed in tap water, colonies counted, and the titre determined.

Freezing and Thawing Retroviral Preparations

Control unconcentrated retrovirus was frozen in 2-5 ml aliquots by placing at −20° C. Concentrated PMP captured retrovirus was frozen by the addition of three volumes of standard freezing mixture (10% DMSO, 20% FCS, final DMSO concentration: 7.5%) and placing at −20° C. Retroviral preparations were thawed as rapidly as possible, and an aliquot removed for testing and the remains returned to −20° C.

Magnetic Enhancement of Retroviral Infection

HeLa cells were plated at 2×10⁶ cells/90 mm dish and cultured overnight at 37° C. Prior to infection the magnetic shape required (cut from Bisiflex II sheets) was taped to the underside of the culture dish, the culture medium was also adjusted to 4 μg/ml Polybrene and 2-4 hours later 7.5×10⁶ magnetic particles loaded with biotinylated PG13 derived retrovirus was added in 5 ml of fresh medium. The cultures were then agitated (40 cycles/minute) for 30 minutes at room temperature (The Belly Dancer, Stovall Life Sciences Inc, Greenboro, N.C., USA) after which the cultures were placed at 37° C. After 24 hours the magnet was removed, the medium changed and the culture returned to 37° C. After a total of 48 hours infection the medium was adjusted to 5 μg/ml puromycin and after daily medium changes (maintained drug selection) the cultures were stained (72 hours after initiation of selection) with Commasie blue stain (5.35% wt/vol in 45% methanol: 10% acetic acid).

References

1 Miller A D, Rosman G J. Biotechniques 1989; 8: 980-990.

2 Riviere I, Brose K, Mulligan R C. Proc. Natl. Acad. Sci. USA 1995 92: 6733-6737.

3 Miller A D et al. J. Virol. 1991; 65: 2220-2224.

4 Ory D S, Neugeboren B A, Mulligan R C. Proc. Natl. Acad. Sci. USA 1996; 93: 11400-11406.

5 Cosset F -L et al. J. Virol. 1995; 69: 7430-7436.

6 Burns J C et al. Proc. Natl. Acad. Sci. USA 1993; 90: 8033-8037.

7 Kozak S L, Kabat D. J. Virol. 1990; 64: 3500-3508

8 Parente M K, Wolfe J H. Gene Therapy 1996; 3: 756-760.

9 Kotani H et al. Hum. Gene Ther. 1994; 5: 19-28

10 Bunnell B A et al. Proc. Natl. Acad. Sci. USA 1995; 92: 7739-7743.

11 Olsen J C, Sechelski J. Hum. Gene Ther. 1995; 6: 1195-1202.

12 Manning J S, Hackett A J, Darby-Jr N B. Appl. Microbiol. 1971; 22: 1162-1163.

13 Toyoshima K, Vogt P K. Virology 1969; 38: 414-426

14 Porter C D et al. J. Virol. 1998; 72: 4832-4840.

15 Chuck A S, Clarke M F, Palson B O. Hum. Gene Ther. 1996; 7: 1527-1534.

16 Hanenberg H et al. Nature Med. 1996; 2: 876-882.

17 Paul R W et al. Hum. Gene Ther. 1993; 4: 609-615.

18 Miller D L, Meikle P J, Anson D S. Nucl. Acids Res. 1996 24: 1576-1577.

19 Chu T-H T, Dornburg R. J. Virol. 1997; 71: 720-725.

20 Olsen J C, Johnson L G, Yankaskas J R. In: Robbins P (ed). Gene Therapy Protocols. Humana Press:New Jersey, 1997, pp153-168

21 Morling F J, Russell S J. Gene Therapy 1995; 2: 504-508.

22 LeDoux J M, Morgan J R, Snow R G, Yarmush M L. J. Virol. 1996; 70: 6468-6473.

23 Russell S J. In: Collins MKL (ed). Methods in Molecular Biology: Vol 8 Practical Molecular Virology. Humana Press:New Jersey, 1991, pp29-48

24 Bowles N E et al. Hum. Gene Ther. 1996; 7: 1735-1742.

25 Seppen J, Kimmel R J, Osborne WRA. Biotechniques. 1997; 23: 788-790.

26 McNeish I A et al. Gene Therapy 1998; 5: 1061-1069.

27 Liu M -L, Winther B L, Kay M A. J. Virol. 1996; 70 :2497-2502.

28 Palson B, Andreadis S. Exp. Haem. 1997; 25: 94-102.

29 Morgan J R et al. J. Virol. 1995; 69: 6994-7000.

30 Gäken J et al. (submitted for publication)

31 Dardalhon V et al. Hum. Gene Therap. 1999; 10: 5-14

32 Signas C et al. Proc. Natl. Acad. Sci. USA. 1989; 86: 699-703.

33 Jonsson K et al. Eur. J. Biochem. 1991; 202: 1041-1048.

34 Shantz E M. PANSORBIN, Calbiochem:1994.

35 Freeman G T et al. J. Immunol. 1989; 143: 2714-2722.

36 Spear G T et al. J. Immunol. 1995; 155: 4376-4381.

37 Balliet J W, Bates P. J. Virol. 1998; 72: 671-676.

38 Izumi M et al. Exp. Cell Res. 1991; 197: 229-233

39 Markowitz D, Goff S, Bank A. Virology 1988; 167: 400-406.

40 Miller A D, Buttimore C. Mol. Cell Biol. 1986; 6: 2895-2902

41 Markowitz D, Goff S, Bank A. J. Virol. 1988; 62: 1120-1124.

42 Kayman S C et al. J. Virol. 1999; 73: 1802-1808.

43 Daley G Q, Baltimore D. Proc. Natl. Acad. Sci. USA. 1988; 85: 9312-9316.

44 Vercellotti G M et al. Am. J. Path. 1985; 120: 13-21.

45 Jordan-Sciutto K L et al. Exp. Cell Res. 1997; 236: 527-536.

46 Dean D C, Newby R F, Bourgeois S. J. Cell Biol 1988; 106: 2159-2170.

47 Gerin P A et al. Hum. Gene Therap. 1999 10: 1965-1974

48 Touhami M, Bourge J -F, Legrand C. Brit. J. Haem. 1999; 104: 706-714.

49 Bansal S C et al. Cancer 1978; 42: 1-18.

50 Cowan F M. Toxicol. Meth. 1997; 7: 9-15.

51 Steele G, Ankerst J, Sjogren H O. Int. J. Cancer 1974; 14: 83-92.

52 Fennelly D W et al. Cancer 1995; 75: 2099-2102.

53 Messerschmidt G L et al. Semin. Haem. 1989; 26 (suppl 1): 19-24.

54 Engelman R W, Good R A, Day N K. Cancer Detect. Prev. 1987; 10: 435-444.

55 Snyder H W, Reed D E, Jones F R. Semin. Haem. 1989; 26 (suppl 1): 25-30.

56 Darling, D., Hughes, C. Galea-Laun, J. Gäken, J. Trayner, I D., Kuiper, M and Farzaneh F. 2000 Gene. Therapy (in press).

57 Takeuchi, Y., Porter, C. D., Strahan, K. M. Preece, A. F., Gustafsson, K. Cosset, F -L. et al. Nature, 1996, 379:85-88.

58 Larsen, R. D., et al 1990, J. Biol. Chem, 265: 7055-7061.

59 Chuck A S, Clarke M F, Palson B O. Hum. Gene Ther. 1996; 7: 1527-1534.

60 Galili, U. and Swanson, K. 1991, Proc. Natl. Acad. Sci. USA. 88: 7401-7404.

61 Wood, et al Arch. Biochem. Biophys. 1979, vol 198 p1

62 Reeke G. N. Ann NY Acad Sci vol. 234, 369 1974

63 Animashshaun, T. et al 1993, Antivir. Che. Chemotherap. 4: 145-153

64 Gilbert, M. 1993. J. Virol. Meth 42: 1-12

65 Hayakawa, T. et al 1998. J. Med. Virol. 56: 327-331

66 Cosset, F. L. et al 1995, J. Virol. 69: 7430-7436

67 Yu, H. et al 1998 Virology 243: 415-422

68 Spies C. P. and Compans R. W. 1993 J. Virol. 67: 6535-6541

1. A method of increasing viral titre from a sample comprising viral particles, said method comprising contacting said sample with a binding member capable of binding to the viral particles to form a complex; and concentrating said complex, wherein said complex is capable of infecting a target cell.

2. A method according to claim 1 wherein said complex is concentrated by centrifugation.

3. A method according to claim 1 or claim 2 further comprising the step of determining the viral titre.

4. A method according to any one of the preceding claims wherein said viral particles are retroviral particles.

5. A method according to any one of the preceding claims wherein toe protein is fibronection.

6. A method according to any one of the preceding claims wherein said binding member is a particulate and dense substrate.

7. A method according to claim 3 wherein the particulate and dense substrate is pansorbin.

8. A method according to any one of claims 1 to 5 wherein the method further comprises adding a capture agent capable of capturing the complex.

9. A method according to claim 7 or claim 8 wherein the binding member is an antibody directed against a protein associated with the viral particle, said antibody being associated with a first coupling partner capable to coupling to a second coupling partner associated with the capture agent.

10. A method according to claim 7 or claim 8 wherein the binding member is a lectin capable of binding to glycosylated proteins on the surface of the viral particles, said lectin being associated with a first coupling partner capable of coupling to a second coupling partner associated with the capture agent.

11. A method according to claim 10 wherein the lectin is isolectin B₄ or Succinyl-Concanavalin A.

12. A method according to any one of claim 9 to 11 wherein the coupling partners are selected from the group consisting of biotin/biocytin-avidin/streptavidin, receptor-ligand, and antibody-antigen.

13. A method of modifying a viral particle so as to ease its capture from a sample comprising said modified viral particles so as to increase their titre, said method comprising the steps of incorporating a coupling partner on the surface of a viral packaging cell so that viral particles derived from the packaging cell display said coupling partner on their surface.

14. A method according to claim 13 wherein said coupling partner is biotin co-valently coupled to proteins on the surface of the packaging cell.

15. A method according to claim 14 wherein said biotin is co-valently coupled to the proteins using a succinimide ester.

16. A method according to any one of claims 13 to 15 further comprising the steps of concentrating said modified viral particles using a capture agent comprising a second coupling partner capable of coupling to the coupling partner incorporated on to the surface of the viral particle to form a complex; and concentrating said complex.

17. A method according to any one of claims 1 to 12 and claim 16 wherein said complex is isolated for use in the preparation of a medicament for use in medical treatment.

18. A complex comprising a viral particle and a paramagnetic particle coupled together via a first and a second coupling partner, for use in in vivo targeting.

19. Use of a complex according to claim 18 for use in the preparation of a medicament for in vivo targeting.

20. A method of targeting a complex according to claim 18 to a tissue within a human or animal body, said method comprising the steps of administering the complex to the human or animal body, and drawing the complex to the tissue using a magnetic field.

21. A method according to claim 20 wherein said virus is a retrovirus.

22. A method according to claim 20 or claim 21 wherein said virus comprises an exogenous nucleic acid sequence for expression in the targeted tissue.

23. A modified viral particle produced by a method according to any one of claims 13 to 15 for use in targeting a tissue, said modified viral particle further comprising an antibody or antibody binding domain specific for said tissue, said antibody or antibody binding domain being coupled to the particle via the coupling partner on the viral particle surface.

24. A modified viral particle according to claim 23 which is a retroviral particle.

25. A method of isolating a virus from a sample, said method comprising
contacting said sample with a binding member capable of specifically binding said virus, so that said virus and binding member form a complex;
contacting said complex with a capture agent capable of capturing said complex; and
isolating said complex and capture agent from the sample.

26. A method according to claim 25 wherein said binding member is associated with a first coupling partner capable of coupling to a second coupling partner associated with the capture agent.

27. A method according to claim 26 wherein the coupling partners are biotin and streptavidin.

28. A method according to claim 26 or claim 27 wherein the binding member is an antibody and the capture agent is a paramagnetic particle.

29. A method according to claim 28 wherein the complex and capture agent are isolated from the sample using a magnetic field.

30. A method according to claim 26 or claim 27 wherein the capture agent is on a solid support over which the sample is passed leaving the complex and capture agent isolated on the solid support.

31. A method according to any one of claims 25 to 27 wherein the binding member is a lectin.

32. A method according to any one of claims 25 to 31 wherein the sample is blood, urine, serum or semen.

33. A method according to any one of claims 25 to 32 wherein the virus is HIV.

34. A kit for carrying out a method according to any one of claims 1 to 12 comprising
(i) a binding member capable of binding to a virus to form a complex; and optionally
(ii) a capture agent capable of capturing the complex via a coupling partner.
